(19) United States
(12) Patent Application Publication
(10) Pub. No.: US 2026/0321399 A1
(43) Pub. Date: Oct. 15, 2026

(54) METHOD AND DEVICE FOR PREDICTING TREATMENT RESPONSE TO HER2-TARGETED THERAPEUTIC AGENT

(71) Applicants: LUNIT INC., Seoul (KR); NATIONAL CANCER CENTER, Tokyo (JP)

(72) Inventors: Chan Young OCK, Seoul (KR); Kyung Hyun PAENG, Seoul (KR); Dong Geun YOO, Seoul (KR); Yoshiaki NAKAMURA, Kashiwa-shi (JP); Mitsuho SUMIDA, Kashiwa-shi (JP)

(73) Assignees: LUNIT INC., Seoul (KR); NATIONAL CANCER CENTER, Tokyo (JP)

(21) Appl. No.: 19/485,467

(22) PCT Filed: May 21, 2024
(86) PCT No.: PCT/KR2024/006853

§ 371 Date: Nov. 18, 2025

Related U.S. Application Data
(60) Provisional application No. 63/468,649, filed on May 24, 2023.

(51) Int. Cl. G16H 30/40; G01N 21/64; G06N 3/0464; G06T 7/00

(52) U.S. Cl. CPC G16H 30/40; G01N 21/6486; G06N 3/0464; G06T 7/0012; G01N 2333/82; G01N 2800/52; G06T 2207/30024

(57) ABSTRACT

The present disclosure relates to a method and device for predicting a treatment response to a HER2-targeted therapeutic agent. In an embodiment, a method of more accurately predicting the therapeutic responsiveness of a cancer patient to the HER2-targeted therapeutic agent and treating cancer may be determined.

### TECHNICAL FIELD

The present disclosure relates to a method and device for predicting a treatment response to a HER2-targeted therapeutic agent.

### BACKGROUND ART

Cancer therapeutic agents can be classified into first-generation chemotherapy, which attacks not only cancer cells but also normal cells, a second-generation targeted therapeutic agent, which selectively attacks only cancer cells, and a cancer immunotherapeutic agent, which activates the immune system so that lymphocytes surrounding cancer tissue selectively attack tumor cells.

In particular, human epidermal growth factor receptor 2 (HER2), which is a transmembrane tyrosine kinase receptor, has been shown to be involved in various types of cancer, and thus, cancer therapeutic agents that target HER2 are being developed in various ways. Among these, the development of trastuzumab, which is a humanized anti-HER2 monoclonal antibody (mAb), has significantly improved clinical outcomes for HER2-positive breast cancer, and has laid the foundation for further development of HER2-targeting approaches, including tyrosine-kinase inhibitors, novel monoclonal antibodies (mAbs), antibody-drug conjugates such as trastuzumab deruxtecan, and the like.

However, cancer therapeutic agents may cause side effects and are not always effective for cancer patients. In addition, therapies such as chemotherapy or targeted therapy cause physical and/or mental pain in cancer patients. Therefore, before therapy such as immunotherapy or targeted therapy is applied to cancer patients, it is important to determine whether these therapies are effective for patients, i.e., determine whether all or at least part of tumors of cancer patients can be removed.

Meanwhile, current HER2-targeted therapy uses HER2 as a biomarker and uses the expression level of a HER2 protein in tumor tissue as a major criterion for predicting the responsiveness of cancer patients to HER2-targeted therapy. Specifically, HER2-targeted therapy involves collecting tissue from a patient before the therapy, and then straining the tissue through immunohistochemistry (IHC), manually calculating the expression level of the HER2 protein in a stained area by a pathologist, and administering a HER2-targeted therapeutic agent to patients whose expression level is a certain expression level or greater. However, one IHC stained whole slide image can contain 1,000 to 1,000,000 tumor cells, and a process of quantifying cells with different staining intensities depending on the expression level of the HER2 protein is labor-intensive and time-consuming. Thus, it is not easy to objectively, accurately and rapidly quantify HER2 expression without the help of a computing system.

The present disclosure provides the prediction of a treatment response to a HER2-targeted therapeutic agent, performed by a computing device, i.e., an AI-powered IHC analyzer, and a method of improving the prediction of treatment responsiveness of a patient to a HER2-targeted therapeutic agent according to the HER2 expression level and/or tumor microenvironment (TME) factors of a cancer patient, by using the computing device.

### DISCLOSURE

### Technical Problem

The objective of the present disclosure is to provide a method and device for predicting a treatment response to a HER2-targeted therapeutic agent. Also, the objective of the present disclosure is to provide a computer-readable recording medium that stores a program for executing the method in a computer. Technical problems to be solved are not limited to the above-described technical problems, and may include other technical problems.

### Technical Solution

According to one aspect, a method for predicting a treatment response to a HER2-targeted therapeutic agent includes identifying the HER2 staining intensities of tumor cells (TC) in a first pathology slide image of a patient by using a machine learning model, determining the proportion of tumor cells exhibiting HER2 IHC 3+ (H3) staining intensity on the basis of the identified results, and predicting a treatment response of the patient to a HER2-targeted therapeutic agent on the basis of the proportion of tumor cells exhibiting HER2 IHC 3+ (H3) staining intensity.

According to another aspect, a method for providing information of the prediction of a treatment response to a HER2-targeted therapeutic agent includes identifying the HER2 staining intensities of tumor cells (TC) in a first pathology slide image of a patient by using a machine learning model, determining the proportion of tumor cells exhibiting HER2 IHC 3+ (H3) staining intensity on the basis of the identified results, and predicting a treatment response of the patient to a HER2-targeted therapeutic agent on the basis of the proportion of tumor cells exhibiting HER2 IHC 3+ (H3) staining intensity.

According to another aspect, a computer-readable recording medium includes a recording medium that stores a program for executing the above-described method in a computer.

According to another aspect, a computing system includes at least one memory, and at least one processor connected to the memory and configured to execute at least one program that is readable by a computer included in the memory.

The at least one processor is configured to, by executing the at least one program, identify the HER2 staining intensities of tumor cells (TCs) in a first pathology slide image of a patient by using a machine learning model, determine the proportion of tumor cells exhibiting HER2 IHC 3+ (H3) staining intensity on the basis of the identified results, and predict a treatment response of the patient to a HER2-targeted therapeutic agent on the basis of the proportion of tumor cells exhibiting HER2 IHC 3+ (H3) staining intensity.

According to another aspect, there is provided a method of treating cancer, including identifying the HER2 staining intensities of tumor cells (TCs) in a first pathology slide image of a patient by using a machine learning model, determining the proportion of tumor cells exhibiting HER2 IHC 3+ (H3) staining intensity on the basis of the identified results, predicting a treatment response of the patient to a HER2-targeted therapeutic agent on the basis of the proportion of tumor cells exhibiting HER2 IHC 3+ (H3) staining intensity, and administering the HER2-targeted therapeutic agent to the patient when it its determined that the patient responds to the HER2-targeted therapeutic agent, on the basis of the results of prediction of the treatment response.

### Advantageous Effects

A method or device for predicting a treatment response to a HER2-targeted therapeutic agent, according to an aspect, can be used to more accurately predict the therapeutic responsiveness of a cancer patient to a HER2-targeted therapeutic agent and determine a treatment method.

### DESCRIPTION OF DRAWINGS

FIG. 1 is a view for explaining an example of a computing system that generates the results of analysis of pathological images, according to an embodiment.

FIG. 2 is a configuration diagram illustrating an example of a computing system for predicting a treatment response to a HER2-targeted therapeutic agent, according to an embodiment.

FIG. 3 is a diagram for explaining examples of the analysis of pathological images by a processor according to an embodiment.

FIG. 4 is a flowchart for explaining an example of a method of predicting a treatment response to a HER2-targeted therapeutic agent, according to an embodiment.

FIG. 5 illustrates the results of confirming the HER2 status of colorectal cancer patients as HER2 IHC scores and proportions of HER2 gene amplification of pathologic slides of the patients.

FIG. 5A illustrates the results of evaluating HER2 IHC scores in Prescreening cohort (n=144) and TRIUMPH cohort (n=30) by using an AI-powered HER2 quantification continuous score (QCS) analyzer, and FIG. 5B illustrates the results of of analyzing proportions of HER2 amplification through the results immunohistochemistry (IHC) and fluorescence in situ hybridization (FISH) or the results of circulating tumor DNA analysis (ctDNA) in the TRIUMPH study, in which the status of HER2 overexpression or gene amplification indicated as HER2 positive (HER2+).

FIG. 6 is a graph showing the results of analyzing the HER2 status of patients enrolled in the TRIUMPH trial by using HER2 IHC, FISH and ctDNA analysis results and the proportion (AI-H3 prop, %) of tumor cells exhibiting HER2 IHC 3+ (H3) staining intensity determined by an AI model (the x-axis represents HER2 immunohistochemistry (IHC) scores evaluated by pathologists, and the y axis represents a HER2/CEP17 ratio determined through fluorescence in situ hybridization (FISH).

FIG. 6A illustrates HER2+ (positive), HER2-(negative), or an unexamined case, which are determined through HER2 ctDNA analysis, with colored dots, and FIG. 6B illustrates the proportion (AI-H3 prop, %) of tumor cells exhibiting HER2 IHC 3+ (H3) staining intensity, determined by the AI model, with colored dots.

FIG. 7 is a graph showing the results of evaluating the proportion (AI-H3 prop) of tumor cells exhibiting HER2 IHC 3+ (H3) staining intensity in the respective pathology slides (WSI) of Prescreening cohort and TRIUMPH cohort by using an AI-powered HER2 QCS analyzer.

FIG. 8 illustrates images showing representative clinical cases of TRIUMPH cohort with low (18.1%) AI-H3 prop and high (97.4%) AI-H3 prop in pathology slides with HER2 IHC 3+ score.

FIG. 8A is an image showing a representative clinical case with low (18.1%) AI-H3 prop in the pathology slide with HER2 IHC 3+ score in the TRIUMPH cohort, and FIG. 8B is an image showing a representative clinical case having high (97.4%) AI-H3 prop in the pathology slide with HER2 IHC 3+ score in the TRIUMPH cohort.

FIG. 9 is a graph showing the proportions of tumor cells (TCs) with HER2 staining intensities of HER2 IHC 0 (H0), HER2 IHC 1+ (H1), HER2 IHC 2+ (H2) and HER2 IHC 3+ (H3) in slides according to HER2 IHC scores classified by an AI-powered HER2 QCS analyzer in the Prescreening cohort and the TRIUMPH cohort.

FIG. 10 illustrates the results of confirming the agreement between HER2 IHC scores of pathology slides, evaluated for TRIUMPH cohort patients by an AI-powered HER2 QCS analyzer and pathologists.

FIG. 11 is a graph showing the clinical outcomes of groups with total HER2+, Path HER2+, ctDNA HER2+, Path HER2 IHC 3+, AI-H3 prop≥10%, and AI-H3 prop≥50%.

FIG. 12 is a waterfall plot showing tumor regression percentages from the baseline of TRIUMPH trial patients administered with pertuzumab and trastuzumab.

FIG. 13 is a graph showing the correlation between the proportion of tumor cells exhibiting HER2 IHC 3+ (H3) staining intensity, measured by an AI-powered HER2 analyzer, and tumor regression changes of TRIUMPH trial patients administered with pertuzumab and trastuzumab.

FIG. 14 is a set of graphs showing the proportion (AI-H0 prop, AI-H1 prop, AI-H2 prop or AI-H3 prop) of tumor cells having each HER2 staining intensity (HER2 IHC 0 (H0), HER2 IHC 1+ (H1), HER2 IHC 2+ (H2), or HER2 IHC 3+ (H3)) in the TRIUMPH study.

FIG. 14A is a set of graphs showing the distribution of proportions (AI-H0 prop, AI-H1 prop, AI-H2 prop, and AI-H3 prop) of tumor cells having HER2 staining intensity of HER2 IHC 0 (H0), HER2 IHC 1+ (H1), HER2 IHC 2+ (H2) or HER2 IHC 3+ (H3) in TRIUMPH cohort slides, in which the slides were classified by HER2 IHC scores, and FIG. 14B is a graph showing the proportions (AI-H0 prop, AI-H1 prop, AI-H2 prop and AI-H3 prop) of tumor cells having HER2 staining intensities of HER2 IHC 0 (H0), HER2 IHC 1+ (H1), HER2 IHC 2+ (H2), and HER2 IHC 3+ (H3) for each TRIUMPH cohort case.

FIG. 15 illustrates the overall response rates (ORRs) of progression-free survival (PFS) and overall survival (OS) and Kaplan-Meier curves, according to AI-H3 prop<50% or AI-H3 prop≥50%.

FIG. 16 is a set of graphs showing the results of comparative analysis of AI-powered tumor microenvironment factors (lymphocyte (LC) density, macrophage (MP) density, fibroblast (FB) density, and endothelial cell (EC) density) between responders and non-responders of TRIUMPH patients.

FIG. 17 is a set of graphs showing the results of comparison in each TME factor between a group with AI-H3 prop<50% and a group with AI-H3 prop≥50% in the TRIUMPH cohort.

FIG. 18 is a set of graphs showing the results of comparison in TME factors between responders (patients who achieved complete response (CR) or partial response (PR) according to RECIST V1.1 criteria) and non-responders (patients who developed stable disease (SD) or progressive disease (PD), the TME factors being quantified by an AI-powered TME analyzer in a group with AI-H3 prop≥50% of the TRIUMPH cohort.

FIG. 19 illustrates Kaplan-Meier curves showing progression free survival (PFS) according to the quantitative proportion (AI-H3 prop) of tumor cells exhibiting AI-powered HER2 IHC 3+ (H3) staining intensity and AI-powered tumor microenvironment factors.

FIG. 19A is a Kaplan-Meier curve for AI-H3 prop and the lymphocyte density of a cancer stroma region (LC-CS), FIG. 19B is a Kaplan-Meier curve for AI-H3 prop and the macrophage density of a cancer stroma region (MP-CS), FIG. 19C is a Kaplan-Meier curve for AI-H3 prop and the fibroblast density of a cancer stroma region (FB-CS), and FIG. 19D is a Kaplan-Meier curve for AI-H3 prop and endothelial cell density (EC) in a cancer stroma region (EC-CS).

FIG. 20 illustrates Kaplan-Meier curves showing overall survival (OS) according to the quantitative proportion (AI-H3 prop) of tumor cells exhibiting AI-powered HER2 IHC 3+ (H3) staining intensity and AI-powered tumor microenvironment factors.

FIG. 20A is a Kaplan-Meier curve for AI-H3 prop and the lymphocyte density of a cancer stroma region (LC-CS), FIG. 20B is a Kaplan-Meier curve for AI-H3 prop and the macrophage density of a cancer stroma region (MP-CS), FIG. 20C is a Kaplan-Meier curve for AI-H3 prop and the fibroblast density of a cancer stroma region (FB-CS), and

FIG. 20D is a Kaplan-Meier curve for AI-H3 prop and endothelial cell density (EC) in a cancer stroma region (EC-CS).

FIG. 21 illustrates graphs showing hazard ratios (HR) according to lymphocyte density (LC-CS), fibroblast density (FB-CS), macrophage density (MP-CS), and endothelial cell density (EC-CS), in cancer stroma (CS) regions of patients having a quantitative proportion of tumor cells exhibiting AI-powered HER2 IHC 3+ (H3) staining intensity of 50% or more (AI-H3 prop≥50%).

FIG. 21A is a graph showing the hazard ratio (HR) for PFS in patients having a quantitative proportion of tumor cells exhibiting AI-powered HER2 IHC 3+ (H3) staining intensity of 50% or more (AI-H3 prop≥50%), and FIG. 21B is a graph showing the HR for OS in patients having a quantitative proportion of tumor cells exhibiting AI-powered HER2 IHC 3+ (H3) staining intensity of 50% or more (AI-H3 prop≥50%).

FIG. 22 illustrates graphs showing hazard ratios (HR) according to lymphocyte density (LC-CA), fibroblast density (FB-CA), macrophage density (MP-CA), and endothelial cell density (EC-CA), in cancer areas (CAs) of patients having a quantitative proportion of tumor cells exhibiting AI-powered HER2 IHC 3+ (H3) staining intensity of 50% or more (AI-H3 prop≥50%).

FIG. 22A is a graph showing the hazard ratio (HR) for PFS in patients having a quantitative proportion of tumor cells exhibiting AI-powered HER2 IHC 3+ (H3) staining intensity of 50% or more (AI-H3 prop≥50%), and FIG. 22B is a graph showing the HR for OS in patients having a quantitative proportion of tumor cells exhibiting AI-powered HER2 IHC 3+ (H3) staining intensity of 50% or more (AI-H3 prop≥50%).

FIG. 23 illustrates Kaplan-Meier curves showing the progression-free survival (PFS) and overall survival (OS) of a combination of LC-CS, MP-CS, and FB-CS.

FIG. 24 illustrates representative clinical cases of a group with TME-high or TME-low among groups having a proportion of tumor cells exhibiting HER2 IHC 3+ (H3) staining intensity of 50% or more (AI-H3 prop≥50%) in the TRIUMPH cohort.

FIG. 24A illustrates a representative clinical case exhibiting a PD treatment response in an AI-H3 prop≥50% with TME-high group, and FIG. 24B illustrates a representative clinical case exhibiting a CR treatment response in an AI-H3 prop≥50% with TME-low group.

FIG. 25 is a nomogram for estimating 12-month and 24-month overall survival (OS) rates of patients having a proportion of tumor cells exhibiting HER2 IHC 3+ (H3) staining intensity of 50% or more (AI-H3 prop≥50%).

FIG. 26 is a graph showing the correlation between factors analyzed in HER2 IHC WSIs and H&E WSIs through an AI-powered HER2 QCS analyzer and an AI-powered TME analyzer.

### MODE FOR INVENTION

The terms used in the embodiments are selected, as much as possible, from general terms that are widely used currently, but these terms may vary depending on intentions of those of ordinary skill in the art, precedents, emergence of new technologies, or the like. Also, in a particular case, terms that are arbitrarily selected by the applicant of the present disclosure may be used. In this case, the meanings of these terms may be described in corresponding description parts of the disclosure. Accordingly, it should be noted that the terms used herein should be construed based on practical meanings thereof and the whole content of this specification, rather than being simply construed based on names of the terms.

In the entire specification, when an element is referred to as “including” another element, the element should not be understood as excluding other elements unless otherwise specifically stated, and the element may further include other elements. In addition, the terms “unit,” “module,” “part,” and the like denote a unit processing at least one function or operation, and may be implemented in hardware or software, or may be implemented in a combination of hardware and software.

According to an embodiment of the present disclosure, module or part may be implemented as a processor and a memory. The processor should be interpreted broadly to include a general-purpose processor, a central processing unit (CPU), a graphic processing unit (GPU), a microprocessor, a digital signal processor (DSP), a controller, a microcontroller, a state machine, or the like. Under some circumstances, the “processor” may refer to an application-specific integrated circuit (ASIC), a programmable logic device (PLD), a field-programmable gate array (FPGA), or the like.

The term “each of a plurality of A” used herein may refer to each of all components included in the plurality of A or each of some components included in the plurality of A. For example, each of a plurality of tumor cells may refer to each of all tumor cells included in the plurality of tumor cells or may refer to each of some tumor cells included in the plurality of tumor cells. Similarly, each of a plurality of immune cells may refer to each of all immune cells included in the plurality of immune cells or each of some immune cells included in the plurality of immune cells.

The term “similar” as used herein may encompass the meanings of “same” or “similar.” For example, the similarity of two pieces of information may mean that the two pieces of information are the same as or similar to each other.

The term “instruction” as used herein is a series of commands bundled together based on function, is a component of a computer program, and is executed by a processor.

Hereinafter, embodiments of the present disclosure will be described in detail with reference to the accompanying drawings. However, the embodiments may be implemented in various different forms and are not limited to the examples described herein.

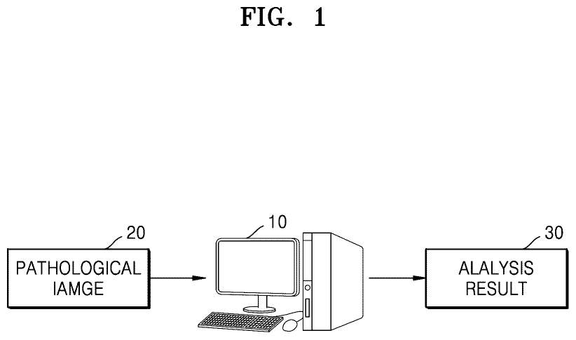

FIG. 1 is a view for explaining an example of a computing system (10) that generates the results of analysis of a pathological image (20), according to an embodiment.

Referring to FIG. 1, the computing system 10 may receive the pathological image 20 and generate an analysis result 30 of the pathological image 20. In this regard, the analysis result 30 and/or medical information generated on the basis of the analysis result 30 may be used to predict the treatment response of a cancer patient to a HER2-targeted therapeutic agent.

FIG. 1 illustrates the computing system 10 as one computing device, but the present disclosure is not limited thereto, and the computing system 10 may be configured to distributedly process information and/or data through a plurality of computing devices. In addition, although FIG. 1 does not illustrate a storage system capable of communicating with the computing system 10, the computing system 10 may be configured to be connected or communicable with at least one storage system. The computing system 10 may be any computing device used to generate the analysis result 30 of the pathological image 20. In this regard, the computing device may refer to any type of device equipped with a computing function, for example, a laptop, a desktop, a laptop, a server, a cloud system, or the like, but the present disclosure is not limited thereto.

A storage system configured to be communicable with the computing system 10 may be a device or a cloud system that stores and manages various data related to pathology image analysis work. For efficient management of data, a storage system may store and manage various data by using a database. In this regard, the various data may include any data related to pathological image analysis, and for example, the various data may include the pathological image 20 and histological components on the type, position, state, and the like of a cell, tissue, and/or structure included in the pathological image 20. In addition, the various data may include clinical factors such as age, menopause status, clinical T-stage (Clinical_T), BIRADS, number of tumors, tumor size, Node_Enlargement, Biopsy_ER, Biopsy_PR, Biopsy_HER2, pCR_final, pathology type, homologous recombination deficiency (HRD), and the like.

The computing system 10 may receive the pathological image 20 obtained from human tissue of a patient that is a subject for predicting a treatment response to a HER2-targeted therapeutic agent. The pathological image 20 may be received via a communicable storage medium (e.g., a hospital system, a local/cloud storage system, or the like). The computing system 10 may analyze the received pathological image 20 and generate the analysis result 30 of the pathological image 20. In this regard, the pathological image 20 may include histological components about at least one patch included in the image.

The term “patch” as used herein may refer to a small region within the pathology image. For example, the patch may include a region corresponding to a semantic object extracted by performing segmentation on the pathology image. As another example, the patch may refer to a combination of pixels associated with histological components generated by analyzing the pathology image.

The term “histological components” as used herein may include characteristics or information about cells, tissues, and/or structures in the human body included in the pathology image. In this regard, the characteristics of cells may include cytologic features such as nucleus, cytoplasm, and cell membrane. The histological components may refer to the histological components of at least one patch included in the pathology image, which are inferred through a machine learning model. The term “annotation” as used herein refers to an operation of tagging histological components to a data sample, or to tagged information (i.e., annotation) itself. The annotation may be used interchangeably with terms such as tagging, labeling, and the like as used in the art.

According to an embodiment, the computing system 10 may analyze the pathological image 20 to extract histological components, which are features of cell, tissue, and/or structure in the body of a target patient. Specifically, the computing system 10 may analyze (e.g., infer) the pathological image 20 by using a machine learning model to extract histological components of at least one patch included in the pathological image 20. For example, the histological components may include, but are not limited to, components (e.g., the number of specific cells and information on tissue in which the specific cells are placed) for cells in the patch (e.g., tumor cells, lymphocytes, macrophages, dendritic cells, fibroblasts, endothelial cells, and the like). In this regard, tumor cells may refer to cells that ignore the cell growth cycle and continue to proliferate excessively. In particular, malignant tumor cells that penetrate (infiltrate) surrounding tissues and spread to distant tissues and grow (metastasize) may be referred to as cancer cells.

According to an embodiment, the computing system 10 may detect the expression of a biomarker in cells included in the pathological image 20 or extract information related to the expression of the biomarker from the pathological image 20. For example, the pathological image 20 may include an image stained by immunohistochemistry (IHC). In addition, the computing system 10 may extract information about the expression level of a biomarker of interest from the stained pathological image 20. For example, the computing system 10 may identify HER2 staining intensities from the pathological image 20. The computing system 10 may calculate a quantitative continuous score (QCS), which represents the proportion of tumor cells (TCs) showing specific HER2 staining intensity, from the pathological image 20. In addition, the computing system 10 may predict the treatment response of a cancer patient to a HER2-targeted therapeutic agent by using the calculated QCS. For example, the computing system 10 may predict the treatment response of a cancer patient to a HER2-targeted therapeutic agent on the basis of the QCS, which represents the proportion of tumor cells exhibiting HER2 IHC 3+ staining intensity.

Other examples of the histological components extracted from a patch by the computing system 10 may include information about tissue in the patch (e.g., a cancer area, cancer epithelial, cancer stroma, normal epithelial, normal stroma, necrosis, fat, background, and the like). Another example of histological components extracted from a patch by the computing system 10 may include information about tumor microenvironment factors (TME factors). Examples of tumor microenvironment factors (TME factors) may include, but are not limited to, lymphocyte (LC) density, macrophage (MP) density, fibroblast (FB) density, and endothelial cell (EC) density. The histological components extracted from a patch are not limited to the above examples, and may include any histological components that can be quantified in the patch, such as cell instability, cell cycle, and biological function.

As described above, the analysis result 30 generated by the computing system 10 and/or medical information generated/output based on the analysis result 30 may be used to predict and/or output the result of treatment response to a HER2-targeted therapeutic agent. In addition, by using patient's clinical information related to pathology slides received from an accessible external system as additional input data, a prediction result of the treatment response of a cancer patient to a HER2-targeted therapeutic agent may be inferred and/or output.

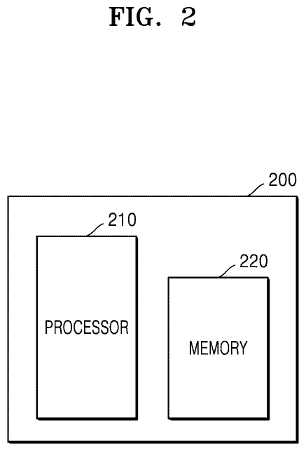

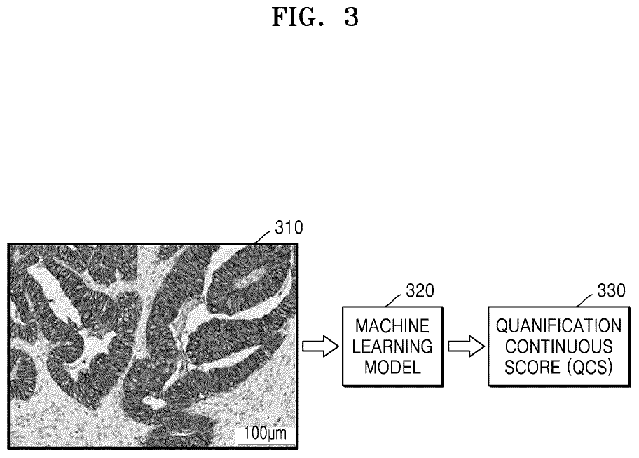

Hereinafter, examples of the analysis of a pathological image by the computing system 10 will be described with reference to FIGS. 2 to 4.

FIG. 2 is a configuration diagram illustrating an example of a computing system 200 for predicting treatment response to a HER2-targeted therapeutic agent, according to an embodiment.

Referring to FIG. 2, the computing system 200 includes a processor 210 and a memory 220. For convenience of explanation, only components related to the present disclosure are illustrated in FIG. 2. Thus, the computing system 200 may further include other general-purpose components, in addition to the components illustrated in FIG. 2. For example, the computing system may include at least one of a server device and a cloud device, but the present disclosure is not limited thereto. As another example, the computing system may include at least one server device. As another example, the computing system may include at least one cloud device. As another example, the system may include both a server device and a cloud device and be operated. In addition, it is obvious to those of ordinary skill in the art that the processor 210 and memory 220 illustrated in FIG. 2 may be implemented as independent devices.

The processor 210 may be configured to process instructions of a computer program by performing basic arithmetic operations, logic operations, and input/output operations. In this regard, the instructions may be provided from the memory 220 or an external device (e.g., a server 20 or the like). In addition, the processor 210 may generally control the operations of other components included in the computing system 200.

The processor 210 may be implemented as an array of multiple logic gates, or may be implemented as a combination of a general-purpose microprocessor and a memory storing a program that may be executed on the microprocessor. For example, the processor 210 may include a general-purpose processor, a central processing unit (CPU), a microprocessor, a digital signal processor (DSP), a controller, a microcontroller, a state machine, and the like. Under some circumstances, the processor 210 may include an application specific integrated circuit (ASIC), a programmable logic device (PLD), a field programmable gate array (FPGA), and the like. For example, processor 210 may refer to a combination of processing devices, such as a combination of a digital signal processor (DSP) and a microprocessor, a combination of a plurality of microprocessors, a combination of one or more microprocessors combined with a DSP core, or any other combinations of such configurations.

The processor 210 may be configured to analyze pathological images. The analysis of the pathological images may include dividing or detecting certain tissues and/or cells from various tissues and cells expressed on pathology slides, and then converting the divided or detected tissues and/or cells into information that can help make medical decisions. In this regard, the conversion of the divided or detected tissues and/or cells into information that can help make medical decisions may mean extracting features that can be used to classify patient's disease pathology, diagnose cancer, establish a cancer treatment plan, prescribe an anti-cancer agent, or predict the likelihood of disease onset, and in particular, may include predicting the treatment response of a cancer patient to a HER2-targeted therapeutic agent.

For example, the processor 210 may identify tumor cells from a pathological image and identify the HER2 staining intensities of each tumor cell, by using at least one machine learning model. For example, the processor 210 may be configured to acquire information related to HER2 protein expression in a whole slide image (WSI) stained using an immunohistochemical staining (IHC) method and quantify HER2 expression on the basis of the acquired information. More specifically, the processor 210 may be configured to classify the expression level of HER2 (HER2 status) into a plurality of stages according to the level of HER2 staining in tumor cells. In this regard, the plurality of stages may be the most commonly used four stages (0, 1+, 2+, 3+), but the present disclosure is not limited thereto, and may be classified into fewer stages or more stages. Specifically, the processor may be configured to divide the cells identified from the pathological image into five classes: HER2 IHC 0 (H0) tumor cells; HER2 IHC 1+ (H1) tumor cells; HER2 IHC 2+ (H2) tumor cells; HER2 IHC 3+ (H3) tumor cells; and other cells (OT). H0 represents tumor cells (TCs) with no membrane staining, and H1 represents tumor cells (TCs) with incomplete membrane staining with faint or barely perceptible intensity. H2 represents TCs with incomplete lateral or circumferential membrane staining with weak/moderate intensity. H3 represents TCs with complete lateral or circumferential membrane staining with strong intensity. OT represents cells other than tumor cells.

In addition, the processor 210 may be configured to calculate a quantitative continuous score (QCS) representing the proportion of tumor cells according to each HER2 staining intensity, on the basis of the result of identifying, by using at least one machine learning model, the HER2 expression level (HER2 status) of each tumor cell on the basis of the HER2 staining strength. The quantification continuous score (QCS) calculated by the processor 210 may mean deriving the proportion of tumor cells having each HER2 staining intensity (HER2 IHC 0 (H0), HER2 IHC 1+ (H1), HER2 IHC 2+ (H2), HER2 IHC 3+ (H3).

In addition, the processor 210 may be configured to calculate the proportion of tumor cells having each HER2 staining strength, and categorize pathology slides obtained from patient's tissue as HER2 IHC 3+ positive (AI-H3 prop>10%), HER2 IHC 2+ equivocal (AI-H3 prop<10% or AI-H2 prop>10%), HER2 IHC 1+ negative (AI-H1 prop>10%), and HER2 IHC 0 (negative) (AI-H1 prop≤10% or if only H0 cells are present) on the basis of international consensus diagnostic criteria. In this regard, AI-H3 prop represents the proportion (%) of tumor cells representing HER2 IHC 3+ (H3) staining intensity determined by the processor 210 in pathology slides obtained from patient's tissue, AI-H2 prop represents the proportion (%) of tumor cells representing HER2 IHC 2+ (H2) staining intensity determined by the processor 210 in pathology slides obtained from patient's tissue, and AI-H1 prop represents the proportion (%) of tumor cells representing HER2 IHC 1+ (H1) staining intensity determined by the processor 210 in pathology slides obtained from patient's tissue.

The international consensus diagnostic criteria for the HER2 status may refer to guidelines for the interpretation of HER2 IHC test results presented by the Standards and Guidelines Committee (SCO) and the College of American Pathologists (CAP). In this regard, the SCO/CAP guidelines are guidelines for accurate evaluation and reporting of HER2-positive tumors used in pathological examination, and refer to guidelines for accurate interpretation of the HER2 status of tumors through HER2 immunohistochemistry (IHC) evaluation and HER2 fluorescence in situ hybridization (FISH) evaluation.

As a result of analysis, the results of classifying pathology slide images as a case determined that, when the proportion of tumor cells exhibiting HER2 IHC 3+ (H3) staining intensity among all tumor cells is a preset certain value or greater, a patient responds to a HER2-targeted therapeutic agent or the drug sensitivity and treatment prognosis of a cancer patient to a HER2-targeted therapeutic agent is good may be output.

Meanwhile, the processor 210 may be configured to identify tumor microenvironment factors (TME factors) from pathological images by using at least one machine learning model. For example, the processor 210 may be configured to analyze a whole slide image (WSI) stained with H&E to identify various objects such as a cancer area, a cancer stroma area, tumor cells, and immune cells and identify tumor microenvironment factors (TME factors). The tumor microenvironment factors (TME factors) may be measured in a cancer area (CA) or a cancer stroma (CS) area, and may include lymphocyte (LC) density, macrophage (MP) density, fibroblast (FB) density, endothelial cell (EC) density, and the like.

In addition, as a result of analysis, the processor 210 may be configured to output the results of classifying pathology slide images as a case determined that, when at least one of lymphocyte (LC) density, macrophage (MP) density, fibroblast (FB) density, and endothelial cell (EC) density is less than a preset certain value, a patient responds to a HER2-targeted therapeutic agent or the drug sensitivity and treatment prognosis of a patient to a HER2-targeted therapeutic agent is good.

The memory 220 may include any non-transitory computer-readable recording medium. As an example, the memory 220 may include a non-volatile mass storage device such as a random access memory (RAM), a read only memory (ROM), a disk drive, a solid state drive (SSD), or flash memory. As another example, a non-volatile mass storage device such as an ROM, a SSD, flash memory, or a disk drive may be a separate persistent storage device distinguished from memory. In addition, an operating system (OS) and at least one program code may be stored in the memory 220.

These software components may be loaded from a computer-readable recording medium separate from the memory 220. The separate computer-readable recording medium may be a recording medium that can be directly connected to the computing system 200, and may include, for example, a computer-readable recording medium such as a floppy drive, a disk, a tape, a DVD/CD-ROM drive, a memory card, or the like.

Although not illustrated in FIG. 2, the computing system 200 may further include a display device. In other embodiments, the computing system 200 may be connected to an independent display device through wired or wireless communication to transmit and receive data therebetween. For example, pathological images, pathology slide images, analysis information of pathology slide images, medical information, additional information based on medical information may be provided to a user through the display device.

FIG. 3 is a diagram for explaining examples of the analysis of pathological images by a processor according to an embodiment. Hereinafter, examples of the analysis of pathological images by the processor 210 will be described with reference to FIG. 3. Examples, which will be described below with reference to FIG. 3, may all be applied to identify tumor cells, identify the HER2 staining intensities of tumor cells, calculate a quantification continuous score (QCS), determine the proportion of tumor cells exhibiting HER2 IHC 3+ (H3) staining intensity, and derive tumor microenvironment factors (TME factors).

Referring to FIG. 3, the processor 210 may be configured to analyze the pathological image 310 by using a machine learning model 320. For example, the processor 210 may be configured to derive an analysis result of the pathological image 310 including a quantification continuous score 330. Specifically, the analysis result of the pathological image 310 may be derived as the proportion of each tumor cell having each HER2 staining intensity (HER2 IHC 0 (H0), HER2 IHC 1+ (H1), HER2 IHC 2+ (H2), and HER2 IHC 3+ (H3)).

For example, the processor 210 may be configured to output detection results in the form of layers representing tissues of the pathological image 310 by using the machine learning model 320. In this case, the machine learning model 320 may be trained to detect regions corresponding to tissues in reference pathology slide images in the pathological image 310, by using learning data including a plurality of reference pathology slide images and a plurality of reference label information.

In addition, the processor 210 may be configured to classify a plurality of tissues expressed in the pathological image 310. Specifically, the processor 210 may be configured to classify the pathological image 310 into at least one of a cancer area, a cancer stroma area, a necrosis area, and a background area. In other embodiments, the processor 210 may be configured to classify the pathological image 310 into at least one of an invasive cancer area and carcinoma in situ.

However, examples of classifying at least a partial region expressed in the pathological image 310 by the processor 210 are not limited to the foregoing description. In other words, without being limited to the six types of areas described above (a cancer area, a cancer stroma area, a necrosis area, a background area, an invasive cancer area, and carcinoma in situ), the processor 210 may be configured to classify at least one area expressed in the pathological image 310 into a plurality of categories on the basis of various criteria. At least one area expressed in the pathological image 310 may be classified into a plurality of categories according to preset standards or standards set by a user.

In addition, the processor 210 may be configured to analyze the pathological image 310 to classify a plurality of cells expressed in the pathological image 310.

First, the processor 210 may be configured to analyze the pathological image 310 to detect cells from the pathological image 310, and output the detection results in the form of layers representing the cells.

The processor 210 may be configured to output the detection results in the form of layers representing cells of the pathological image 310. In this case, the machine learning model 320 may be trained to detect the locations and types of cells in reference pathology slide images in the pathological image 310, by using learning data including a plurality of reference pathology slide images and a plurality of reference label information.

In addition, the processor 210 may be configured to classify a plurality of cells expressed in the pathological image 310. For example, the processor 210 may be configured to classify a plurality of cells displayed in the pathological image 310 into at least one of tumor cells, lymphocytes, macrophages, fibroblasts, endothelial cells, and other cells.

However, examples of classifying cells displayed in the pathological image 310 by the processor 210 are not limited to the foregoing description. In other words, without limitation to the six types of cells described above (i.e., tumor cells, lymphocytes, macrophages, fibroblasts, endothelial cells, and other cells), the processor 210 may be configured to classify, into a plurality of categories, the cells displayed in the pathological image 310 according to various criteria. The cells displayed in the pathological image 310 may be grouped into a plurality of categories according to preset standards or standards set by a user.

According to the above-described process, the processor 210 may be configured to analyze the pathological image 310 to generate information about at least one subject and/or expression information of a biomarker. For example, the expression information of the biomarker may be, but is not limited to, the HER2 staining intensity of tumor cells or the quantification continuous score (QCS) (330).

In this regard, the machine learning model 320 refers to a statistical learning algorithm implemented based on the structure of a biological neural network or a structure for executing the algorithm.

For example, the machine learning model 320 may represent a model having a problem-solving ability by repeatedly adjusting the weights of synapses by nodes, which are artificial neurons forming a network through synaptic combinations as in the biological neural networks, thus training to reduce errors between an accurate output corresponding to a specific input and a deduced output. For example, the machine learning model 320 may include any probability model, neural network model, and the like that is used in artificial intelligence learning methods such as deep learning.

For example, the machine learning model 320 may be implemented as a multilayer perceptron (MLP) consisting of multiple layers of nodes and connections therebetween. The machine learning model 320 according to the present embodiment can be implemented using one of various artificial neural network model structures including the MLP. For example, the machine learning model 320 may include an input layer that receives an input signal or data from the outside, an output layer that outputs an output signal or data corresponding to the input data, and at least one hidden layer positioned between the input layer and the output layer to receive a signal from the input layer, extract the features, and transmit the features to the output layer. The output layer may receive signals or data from the hidden layer and outputs the received signals or data to the outside.

Therefore, the machine learning model 320 may be trained to receive at least one pathological image 310, and extract information about at least one object (e.g., cells, tissues, structures, and the like) included in the pathological image 310 and/or expression information of a biomarker.

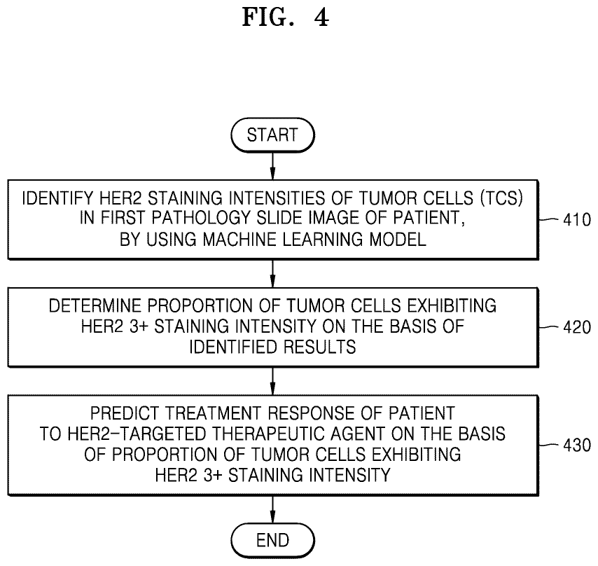

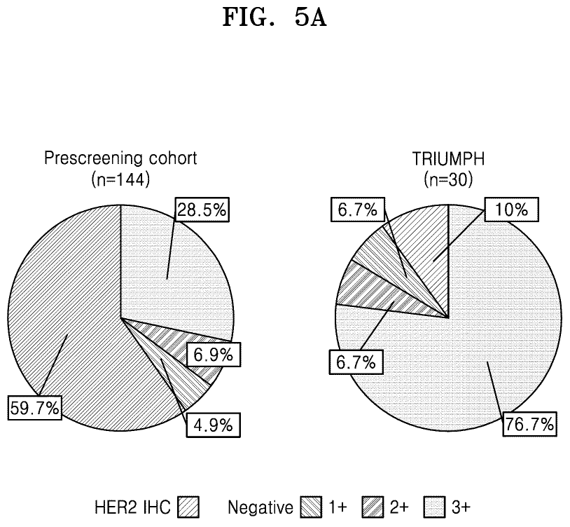

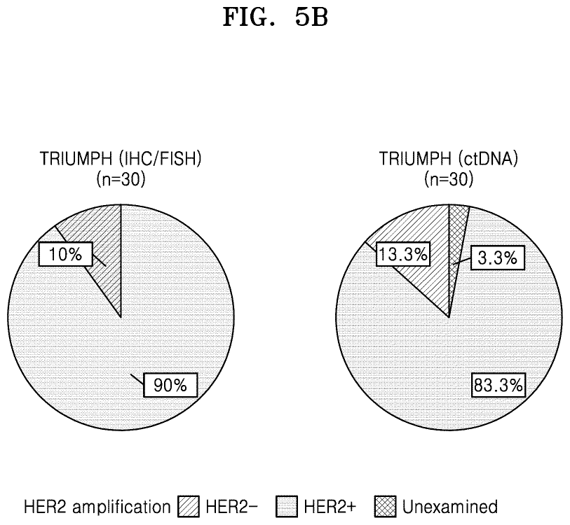

FIG. 4 is a flowchart for explaining an example of a method of analyzing a pathological image, according to an embodiment. The method illustrated in FIG. 4 may include steps processed in time series in the computing systems 10 or 200 or the processor 210 as illustrated in FIGS. 1 and 2. Thus, although detailed description thereof is not provided below, the foregoing description of the computing system 10 or 200 or the processor 210 as illustrated in FIGS. 1 and 2 can also apply to the method illustrated in FIG. 4.

In addition, the method which will be described below may be carried out by a program that can be executed on a computer, and may be implemented on a general-purpose digital computer that operates the program by using a computer-readable recording medium. In addition, the structure of data used in the above-described method may be recorded on a computer-readable recording medium through various means.

The computer-readable recording medium may include a storage medium such as a magnetic storage medium (e.g., ROM, RAM, USB, floppy disk, hard disk, or the like) or an optical readable medium (e.g., CD-ROM, DVD, or the like).

Referring to FIG. 4, in step 410, the processor 210 identifies the HER2 staining intensity of tumor cells (TCs) in a first pathology slide image of a patient by using a machine learning model. The first pathology slide image may refer to a scanned image of a pathology slide fixed and stained through a series of chemical treatments to observe a tissue removed from a human body under a microscope. The pathological image may refer to a whole slide image (WSI) including a high-resolution image of the entire slide, and may include at least one of a hematoxylin & eosin (H&E) staining slide or an immunohistochemistry (IHC) slide, but preferably, may refer to an IHC slide. The first pathology slide image may refer to a portion of an entire high-resolution slide image, for example, one or more patches. In addition, the pathology image may refer to a digital image obtained by scanning a pathology slide by using a digital scanner, and may include information on cells, tissues, and/or structures in the human body.

In an embodiment, the step of identifying the HER2 staining intensites of tumor cells (TCs) in a first pathology slide image of a patient by using a machine learning model may mean acquiring, by the processor 210, information related to HER2 protein expression from a whole slide image (WSI) stained by immunohistochemical staining (IHC), by using at least one machine learning model, and quantifying HER2 expression on the basis of the acquired information. Specifically, the information related to HER2 protein expression may be acquired by classifying, by the processor 210, tumor cells from the first pathology slide image into HER2 IHC 0 (H0), HER2 IHC 1+ (H1), HER2 IHC 2+ (H2), and HER2 IHC 3+ (H3) by using at least one machine learning model. In this regard, H0 represents tumor cells (TCs) with no membrane staining, and H1 represents tumor cells (TCs) with incomplete membrane staining with faint or barely perceptible intensity. H2 represents tumor cells (TCs) with incomplete lateral or circumferential membrane staining with weak/moderate intensity. H3 represents tumor cells (TCs) with complete lateral or circumferential membrane staining with strong intensity.

In step 420, the proportion of tumor cells exhibiting HER2 IHC 3+ (H3) staining intensity is determined based on the results of identifying tumor cells having each HER2 staining intensity (HER2 IHC 0 (H0), HER2 IHC 1+ (H1), HER2 IHC 2+ (H2), and HER2 IHC 3+ (H3)) in the first pathology slide image. Specifically, the proportion of tumor cells exhibiting HER2 IHC 3+ (H3) staining intensity in the first pathology slide image may be derived as a quantification continuous score (QCS).

In step 430, the treatment response of a patient to a HER2-targeted therapeutic agent is predicted based on the proportion of tumor cells exhibiting HER2 IHC 3+ (H3) staining intensity. Specifically, the step of predicting the treatment response of a patient to a HER2-targeted therapeutic agent may include determining that, when the proportion of tumor cells exhibiting HER2 IHC 3+ (H3) staining intensity is 10% or more, the patient responds to the HER2-targeted therapeutic agent. For example, when the processor 210 determines that the proportion of tumor cells (TCs) exhibiting HER2 IHC 3+ (H3) in the first pathology slide image of the patient is 10%, by using at least one machine learning model, the result of classifying the pathology slide image may be output as a case in which the drug sensitivity and treatment prognosis of the patient to the HER2-targeted therapeutic agent are good.

In an embodiment, when the processor 210 determines that the proportion of tumor cells exhibiting HER2 IHC 3+ (H3) staining intensity in a patient is 50% or more, by using at least one machine learning model, it may be determined that the patient responds to the HER2-targeted therapeutic agent.

The method of predicting a treatment response to a HER2-targeted therapeutic agent will be described below in further detail.

According to an aspect, a method of predicting a treatment response to a HER2-targeted therapeutic agent includes the steps of: identifying the HER2 staining intensities of tumor cells (TCs) in a first pathology slide image of a patient by using a machine learning model; determining the proportion of tumor cells exhibiting HER2 IHC 3+ (H3) staining intensity on the basis of the identified results; and predicting a treatment response of the patient to the HER2-targeted therapeutic agent on the basis of the proportion of tumor cells exhibiting HER2 IHC 3+ (H3) staining intensity.

In an embodiment, the method of predicting a treatment response to a HER2-targeted therapeutic agent may include the steps of: calculating the proportions of tumor cells (TCs) having HER2 staining intensities, i.e., HER2 IHC 0 (H0), HER2 IHC 1+ (H1), and HER2 IHC 2+ (H2), and HER2 IHC 3+ (H3), from pathology slide images by using a machine learning model; deriving the proportion of tumor cells exhibiting HER2 IHC 3+ (H3) staining intensity as a quantification continuous score (QCS); and predicting the treatment response of a patient to the HER2-targeted therapeutic agent on the basis of the proportion of tumor cells exhibiting HER2 IHC 3+ (H3) staining intensity, derived as a quantification continuous score (QCS).

The term “pathological image” as used herein refers to a scanned image of a pathology slide fixed and stained through a series of chemical treatments to observe a tissue removed from a human body under a microscope.

The pathological image may refer to a whole slide image (WSI) including a high-resolution image of the entire slide, and may include an image of at least one of a hematoxylin & eosin (H&E) staining slide, an immunohistochemistry (IHC) staining slide, or other pathology slides stained by various staining methods. The pathological image may refer to a portion of a high-resolution entire slide image, for example, one or more patches. In addition, the pathology image may refer to a digital image obtained by scanning a pathology slide by using a digital scanner, and may include information on cells, tissues, and/or structures in the human body. In this specification, “pathological image” may be used interchangeably with “pathology image,” “pathology slide image,” “tissue slide image,” “whole slide image (WSI),” and the like. In addition, the term “pathological image” as used herein may also be referred to as “at least a partial region included in a pathology image.”

The term “human epidermal growth factor receptor type 2 (HER2)” as used herein refers to a protein involved in the proliferation-regulating function of cells, and in the case of HER2 overexpression or gene amplification, cells may develop into tumors or cancer.

The term “tumor cell (TC)” as used herein may refer to a cell that ignores the cell growth cycle and continues to proliferate excessively. In particular, malignant tumor cells that penetrate (infiltrate) surrounding tissues and spread to distant tissues and grow (metastasize) may be referred to as cancer cells.

The term “HER2 IHC score” as used herein refers to a score obtained by evaluating the expression level of the HER2 receptor protein of tumor cells in pathology slides obtained from tissue of a patient, by immunohistochemistry (IHC) staining, and categorizing the pathology slides into scores of HER2 IHC 0, HER2 IHC 1+, HER2 IHC 2+, and HER2 IHC 3+ according to the degree of staining of the tumor cells in the pathology slides in accordance with international consensus diagnostic criteria.

Specifically, when the number of tumor cells in which unstained cell membranes are observed exceeds 90% of all tumor cells, the HER2 IHC score of the pathology slides may be determined as 0. In addition, when the number of tumor cells with complete and intense cell membrane staining is 10% or more of all tumor cells, the HER2 IHC score of the pathology slides may be determined as 3.

When the number of tumor cells with complete and intense cell membrane straining is less than 10% of all tumor cells, and the number of tumor cells with complete and intense cell membrane staining or weak-moderate cell membrane staining is 10% or more of all tumor cells, the HER2 IHC score of the pathology slides may be determined as 2.

When the number of tumor cells with complete and intense cell membrane staining is less than 10% of all tumor cells, the number of tumor cells with complete and intense cell membrane staining or weak-moderate cell membrane staining is less than 10% of all tumor cells, and the number of tumor cells with complete and intense cell membrane staining, weak-moderate cell membrane staining, or faint cell membrane staining is 10% or more of all tumor cells, the HER2 IHC score of the pathology slides may be determined as 1.

In all remaining cases in which the HER2 IHC score of the pathology slides is not determined as 1, 2, or 3, the HER2 IHC score may be determined as 0.

The international consensus diagnostic criteria may refer to guidelines for the interpretation of HER2 IHC test results presented by the Standards and Guidelines Committee (SCO) and the College of American Pathologists (CAP). In this regard, the SCO/CAP guidelines are guidelines for accurate evaluation and reporting of HER2-positive tumors used in pathological examination, and refer to guidelines for accurate interpretation of the HER2 status of tumors through HER2 immunohistochemistry (IHC) evaluation and HER2 fluorescence in situ hybridization (FISH) evaluation. The status of HER2 in tumor tissue obtained from tissue of a patient may be determined according to the SCO/CAP guidelines.

For example, when the HER2 IHC score of pathology slides as determined according to the SCO/CAP guidelines is 0, the case may be analyzed as HER2 negative, when the HER2 IHC score of pathology slides is 3 (HER2 IHC 3+), the case may be analyzed as HER2 positive, and when the HER2 IHC score of pathology slides is 1+ (HER2 IHC 1+) or 2+ (HER2 IHC 2+), the case may further be subjected to HER2 fluorescence in situ hybridization (FISH) evaluation and may be analyzed as HER2 negative or HER2 positive. The term “quantification continuous score (QCS)” as used herein refers to a score obtained by acquiring information related to HER2 protein expression in a whole slide image (WSI) stained by immunohistochemical (IHC) staining, and quantifying HER2 expression on the basis of the acquired information. Specifically, the quantification continuous score may refer to a value obtained by deriving the proportion of tumor cells exhibiting HER2 staining intensity, i.e., HER2 IHC 1+ (H1), HER2 IHC 2+ (H2), or HER2 IHC 3+ (H3), on the basis of the results of classifying the HER2 protein expression level of each tumor cell in a pathology slide image obtained from a tissue of a patient by using an AI model as having HER2 staining intensity, i.e., HER2 IHC 0 (H0), HER2 IHC 1+ (H1), HER2 IHC 2+ (H2), or HER2 IHC 3+ (H3), and identifying the tumor cells as tumor cells having each HER2 staining intensity.

For example, in classifying, by using an AI model, cells in pathology slide images acquired from a tissue of a patient into five classes: tumor cells with HER2 IHC 0 (H0), tumor cells with HER2 IHC 1+ (H1), tumor cells with HER2 IHC 2+ (H2), tumor cells with HER2 IHC 3+ (H3), and other cells (OT), the quantification continuous score may refer to a continuous value of the portion of tumor cells having each staining intensity. Tumor cells having the HER2 IHC 0 (H0) staining intensity refer to tumor cells with no cell membrane staining, and tumor cells with the HER2 IHC 1+ (H1) staining intensity refer to tumor cells having incomplete cell membrane staining with faint or barely perceptible intensity. Tumor cells with HER2 IHC 2+ (H2) staining intensity refer to tumor cells with incomplete lateral or circumferential cell membrane staining with weak/moderate intensity, and tumor cells with HER2 IHC 3+ (H3) staining intensity refer to tumor cells with complete lateral or circumferential cell membrane staining with strong intensity. In an embodiment, the HER2 staining intensity of tumor cells (TC) may include HER2 IHC 0 (H0), HER2 IHC 1+ (H1), HER2 IHC 2+ (H2), or HER2 IHC 3+ (H3).

The term “HER2 IHC 3+ (H3) staining intensity” as used herein may mean that the staining intensity corresponds to HER2 IHC 3+ (H3) in a case in which, as a result of analyzing pathology slide images obtained from the tissue of a patient by using an AI-powered HER2 analyzer, a HER2 expression level in each tumor cell in pathology slides is classified as IHC staining intensity (HER2 IHC 0 (H0), HER2 IHC 1+ (H1), HER2 IHC 2+ (H2), or HER2 IHC 3+ (H3)). Specifically, the staining intensity of HER2 IHC 3+ (H3) refers to the degree of complete membrane staining with strong intensity of tumor cells. For example, the staining intensity of HER2 IHC 3+ (H3) may mean the degree of “complete and intense staining” as defined in the SCO/CAP guidelines.

The term “treatment response of a cancer patient” may include pathological complete response, responsiveness to a cancer immunotherapeutic agent, and the like. The term “pathological complete response (hereinafter, pCR)” as used herein may refer to the absence of invasive cancer in a tissue of the human body according to anti-cancer treatment. For example, pathological complete response may mean a state in which all or at least part of tumor cells present in a tissue of the human body are removed as a result of anti-cancer treatment.

The term “biomarker” as used herein may refer to a marker capable of objectively measuring normal or pathological conditions, the degree of response to a drug, and the like.

In an embodiment, based on the proportion of tumor cells (TCs) having specific HER2 staining intensity in a pathology slide image obtained from a tissue of a patient, the HER2 IHC score of the pathology slide image may be determined, and the response of the patient to a HER2-targeted therapeutic agent may be predicted.

For example, when the proportion of cells having HER2 IHC 3+ (H3) staining intensity in a pathology slide image acquired from a tissue of a patient by using an AI-powered HER2 analyzer exceeds 10% (AI-H3 prop>10%), the HER2 IHC score of the pathology slide image may be determined as HER2 IHC 3+, and when the proportion of cells having HER2 IHC 2+ (H2) staining intensity in a pathology slide image acquired from a tissue of a patient is less than 10% or the proportion of cells having HER2 IHC 2+ (H2) staining intensity exceeds 10% (AI-H3 prop<10% or AI-H2 prop>10%), the HER2 IHC score of the pathology slide image may be determined as HER2 IHC 2+ equivocal. In addition, when the proportion of cells having HER2 IHC 1+ (H1) staining intensity in a pathology slide image acquired from a tissue of a patient exceeds 10% (AI-H1 prop>10%), the HER2 IHC score of the pathology slide image may be determined as HER2 IHC 1+, and when the proportion of cells having HER2 IHC 1+ (H1) staining intensity in a pathology slide image acquired from a tissue of a patient is 10% or less (AI-H1 prop≤10%) or only H0 cells are present, the HER2 IHC score of the pathology slide image may be determined as HER2 IHC 0 negative.

The term “immunohistochemistry (IHC) staining” as used herein may refer to a staining method using the principle of reacting an antibody of interest on a tissue to observe the presence or absence of a protein (antigen) in the nucleus, cytoplasm, or cell membrane of a tissue or cell specimen with an optical microscope. Since a product of an antigen-antibody reaction cannot be observed under a microscope as it is, a method of attaching a marker and then developing the color of the marker may be used. As the color coupler, 3-amino-9-ethylcarbazole (AEC), brown 3,3′-diaminobenzidine (DAB), or the like may be used. In addition, to accurately identify the region where a protein is expressed, counterstaining with hematoxylin may be performed after treatment with the color coupler.

In an embodiment, at least one of the steps included in the method may be performed by a machine learning model.

The term “machine learning model” as used herein may refer to the structure of a computer algorithm that learns from data to discover patterns, make predictions, or make decisions. The machine learning model generally performs learning on training data, and then may perform prediction or classification on new data. For example, the machine learning model may include any model used to infer an answer to a given input.

In an embodiment, the machine learning model may include an artificial neural network model including an input layer, a plurality of hidden layers, and an output layer. In this regard, each layer may include one or more nodes. For example, the machine learning model may be trained to infer histological components from a pathology image and/or at least one patch included in the pathology image. In this case, histological components generated through an annotation operation may be used to train the machine learning model. As another example, the machine learning model may be trained to infer responsiveness to therapy for cancer patients, on the basis of an interaction score, characteristics of at least one of a cell, a tissue, or a structure in a pathological image, and/or a clinical factor of a patient. In addition, the machine learning model may include weights associated with a plurality of nodes included in the machine learning model. The weights may include any parameter associated with the machine learning model.

In the present specification, a machine learning model may refer to an artificial neural network model, and an artificial neural network model may refer to a machine learning model. The machine learning model according to the present disclosure may be a model that is trained by using various learning methods. For example, various learning methods, such as supervised learning, unsupervised learning, self-supervised learning, or reinforcement learning, may be used in the present disclosure, but the present disclosure is not limited thereto.

The term “training” as used herein may refer to any process of changing a weight included in a machine learning model by using at least one patch, interaction scores, histological components, and/or clinical factors. In an embodiment, training may refer to a process of changing or updating weights associated with a machine learning model by performing, once or multiple times, forward propagation and backward propagation on the machine learning model by using at least one patch and histological components.

In an embodiment, the HER2-targeted therapeutic agent may include at least one drug selected from the group consisting of pertuzumab, trastuzumab, trastuzumab emtansine, lapatinib, neratinib, afatinib, tucatinib, and pyrotinib.

The term “HER2-targeted therapeutic agent” as used herein may refer to an agent that targets the overexpression of HER2 protein in HER2-positive tumors. One of the HER2-targeted therapeutic agents may be a monoclonal antibody that specifically binds to the HER2 protein present on the surface of tumor cells, but the present disclosure is not limited thereto. In addition, the HER2-targeted therapeutic agent may be used to control or shrink tumors by inhibiting the growth and metastasis of HER2-positive tumors and inducing the death of tumor cells.

The term “pertuzumab” as used herein refers to a monoclonal antibody that belongs to targeted anti-cancer drugs and targets the HER2/neu receptor of cancer cells. It is known that, when used in combination with trastuzumab, pertuzumab effectively blocks HER2 signaling and slow tumor progression.

The term “trastuzumab” as used herein refers to a targeted anti-cancer agent that targets the HER2/neu receptor overexpressed in cancer cells. Trastuzumab has the effect of slowing tumor progression by binding to the HER2 receptor and inhibiting replication of tumor cells.

In an embodiment, the step of predicting the treatment response of a cancer patient to a HER2-targeted therapeutic agent may include a step of determining that, when the proportion of tumor cells exhibiting HER2 IHC 3+ (H3) staining intensity is 10% or more, drug sensitivity and treatment prognosis to the HER2-targeted therapeutic agent are good, and the cancer patient responds to the HER2-targeted therapeutic agent.

In an embodiment, the step of predicting the treatment response of a cancer patient to a HER2-targeted therapeutic agent may include a step of determining that, when the proportion of tumor cells exhibiting HER2 IHC 3+ (H3) staining intensity is 50% or more, drug sensitivity and treatment prognosis to the HER2-targeted therapeutic agent are good, and the cancer patient responds to the HER2-targeted therapeutic agent.

The term “cancer” as used herein refers to a physiological condition in animals that is typically characterized by abnormal or uncontrolled cell growth. The cancer may be associated with, for example, metastasis, interference with the normal function of adjacent cells, release of cytokines or other secretory products at abnormal levels, suppression or exacerbation of inflammatory or immunological responses, neoplasia, premalignant tumors, malignant tumors, surrounding or distal tissues or organs (e.g., lymph nodes), and the like.

In an embodiment, pathology slide images may be obtained from tissue slides collected from solid cancer tissue. Specifically, the solid cancer may be, but is not limited to, at least one selected from the group consisting of lung cancer, skin cancer, stomach cancer, gastrointestinal cancer, intestinal cancer, colorectal cancer, colon cancer, pancreatic cancer, liver cancer, thyroid cancer, uterine cancer, cervical cancer, ovarian cancer, testicular cancer, prostate cancer, breast cancer, and oral cancer. For example, the colorectal cancer may be metastatic and may be in a state of HER2 overexpression or gene amplification, but the present disclosure is not limited thereto.

In an embodiment, the solid tumor may be in a state of HER2 overexpression or gene amplification. In an embodiment, the method may further include a step of determining whether cancer tissue from which tissue slides are collected is in a state of HER2 overexpression or gene amplification.

The term “overexpression or gene amplification” as used herein may refer to an increase in the expression of a gene related to the HER2 protein, caused by an increase in the number of copies of the gene.

In an embodiment, the method for predicting a treatment response to a HER2-targeted therapeutic agent may further include the steps of: deriving tumor microenvironment factors (TME factors) from a pathological image; and predicting a treatment response to a HER2-targeted therapeutic agent by using the tumor microenvironment factors (TME factors).

The term “tumor microenvironment” as used herein may refer to an environment consisting of tissues and cells that surround tumors. For example, the tumor microenvironment may include not only tumor cells themselves, but also surrounding blood vessels, extracellular matrix (surrounding tissues), immune cells, and inflammatory mediators, but the present disclosure is not limited thereto.

The term “tumor microenvironment factors (TME factors)” as used herein may refer to various cells, signaling molecules, and other environmental factors that are expressed or act in the tumor microenvironment. For example, the tumor microenvironment factors (TME factors) may be at least one selected from the group consisting of lymphocyte (LC) density, macrophage (MP) density, fibroblast (FB) density, and endothelial cell (EC) density.

In an embodiment, the tumor microenvironment factors (TME factors) may be measured in a cancer area (CA) or a cancer stroma (CS) area.

The term “cancer area (CA)” as used herein may refer to an area where tumor cells showing invasion are clustered.

The term “cancer stroma (CS) area” may refer to a peripheral area of a tumor region where tumor-related stromal changes, such as formation of fibrous tissue (desmoplasia) or aggregation of lymphoid cells, appear. For example, the cancer stroma (CS) area may include, but is not limited to, fibrous tissue, blood vessels, immune cells, and inflammatory cells.

In an embodiment, the step of predicting a treatment response to a HER2 targeted therapeutic agent may include a step of determining that, when the proportion of tumor cells exhibiting HER2 IHC 3+ (H3) staining intensity is 50% or more, and at least one of the tumor microenvironment factors (TME factors) measured in a cancer stroma (CS) area has a value smaller than the value described below, drug sensitivity and treatment prognosis to the HER2-targeted therapeutic agent are good, and the cancer patient responds to the HER2-targeted therapeutic agent.
Lymphocyte (LC) density: 766.3
Macrophage (MP) density: 26.4
Fibroblast (FB) density: 1790.8
Endothelial cell (EC) density: 88.3

Term “good treatment prognosis” as used herein may mean that a cancer patient has a high chance of survival depending on treatment and follow-up after cancer diagnosis. Specifically, the term “good treatment prognosis” may mean the absence of or reduction in invasive cancer in human tissue due to anti-cancer treatment, and may mean a case where there is a low or no possibility of tumor recurrence.

According to another aspect, a method of treating cancer includes the steps of: identifying the HER2 staining intensities of tumor cells (TCs) in a first pathology slide image of a patient by using a machine learning model; determining the proportion of tumor cells exhibiting HER2 IHC 3+ (H3) staining intensity on the basis of the identified results; predicting a treatment response of the patient to a HER2-targeted therapeutic agent on the basis of the proportion of tumor cells exhibiting HER2 IHC 3+ (H3) staining intensity; and administering the HER2-targeted therapeutic agent to the patient when it its determined that the patient responds to the HER2-targeted therapeutic agent, on the basis of the results of prediction of the treatment response. Specifically, the cancer may be, but is not limited to, at least one selected from the group consisting of lung cancer, skin cancer, stomach cancer, gastrointestinal cancer, intestinal cancer, colorectal cancer, colon cancer, pancreatic cancer, liver cancer, thyroid cancer, uterine cancer, cervical cancer, ovarian cancer, testicular cancer, prostate cancer, breast cancer, and oral cancer.

Hereinafter, the present disclosure will be described in more detail with reference to the following examples. However, these examples are provided for illustrative purposes only and are not intended to limit the scope of the present disclosure.

### Example 1. Cohort Formation

The Prescreeing cohort and TRIUMPH cohort of this study were formed through HER2-Screening study and GOZILA study.

In the HER2-Screening study, HER2 IHC slides were stained using the PATHWAY HER2/neu (4B5) rabbit monoclonal primary antibody (Ventana Medical Systems, Tucson, AZ, US), and FISH analysis was performed using the PathVysion HER-2 DNA probe kit (Abbott Laboratories, IL, US).

The GOZILA study was conducted in patients with metastatic gastrointestinal cancer (mCRC) who had shown disease progression during chemotherapy in the HER2-Screening study. In the GOZILA study, plasma NGS analysis was performed using the Guardant360 at Guardant Health.

In the HER2-Screening study and the GOZILA study, cases with both HER2 IHC slides and H&E slides were selected to form the Prescreening cohort of this study.

In addition, patients confirmed as HER2 positive through tissue and/or ctDNA analysis in the HER2-Screening and GOZILA studies were enrolled to form the TRIUMPH cohort. The TRIUMPH trial was conducted as a multicenter phase 2 clinical trial in Japan, and the ancestry, race, ethnicity, and socioeconomic status of the participants were not collected as part of this study. Detailed characteristics of patients in the TRIUMPH are shown in Table 1.

TABLE 1

| Characteristics | N (%) or mean ± SD (n = 30) |
|---|---|
| Age | 56.2 ± 13.6 |

FAS (Tissue)

| | |
|---|---|
| Accepted | 27 (90.0%) |
| Not accepted | 3 (10.0%) |

FAS (Liquid)

| | |
|---|---|
| Accepted | 25 (83.3%) |
| Not accepted | 5 (16.7%) |

KRAS mutation (by Guardant360)

| | |
|---|---|
| Wild type | 28 (93.3%) |
| Mutant | 1 (3.3%) |
| Unexamined | 1 (3.3%) |

NRAS mutation (by Guardant360)

| | |
|---|---|
| Wild type | 29 (96.7%) |
| Unexamined | 1 (3.3%) |

BRAF mutation (by Guardant360)

| | |
|---|---|
| Wild type | 28 (93.3%) |
| Mutant | 1 (3.3%) |
| Unexamined | 1 (3.3%) |

PIK3CA mutation (by Guardant360)

| | |
|---|---|
| Wild type | 27 (90.0%) |
| Mutant | 2 (6.7%) |
| Unexamined | 1 (3.3%) |

BRAF mutation (by OCP/OCA)

| | |
|---|---|
| Wild type | 22 (73.3%) |
| Mutant | 1 (3.3%) |
| Unexamined | 7 (23.3%) |

PIK3CA mutation (by OCP/OCA)

| | |
|---|---|
| Wild type | 23 (76.7%) |
| Unexamined | 7 (23.3%) |

MSI status

| | |
|---|---|
| MSS | 20 (66.7%) |
| Unexamined | 10 (33.3%) |

The study protocol received approval from the institutional review boards of all participating institutions, including National Cancer Center Hospital East, Aichi Cancer Center Hospital, National Cancer Center Hospital, National Hospital Organization Kyushu Cancer Center, Hokkaido University Hospital, National Hospital Organization Shikoku Cancer Center, and National Hospital Organization Osaka National Hospital. The study was conducted in strict accordance with the protocol, the Ministerial Ordinance on Good Clinical Practice for Drugs, and the Declaration of Helsinki.

### Example 2. Development of AI-Powered Pathology Slide Image Analyzer

### 2-1. Development of AI-Powered HER2 Analyzer

In an embodiment, an AI-powered HER2 analyzer may be used to analyze a HER2 quantification continuous score (QCS). The AI-powered HER2 analyzer according to one embodiment is based on the DeepLabV3+ convolutional neural network architecture and can be developed using the ResNet-34 backbone network.

The AI-powered HER2 analyzer may include two deep learning (DL)-based AI models: a cell detection model; and a tissue segmentation model.

Specifically, the cell detection model was trained using a dataset including 8.13×10⁵ tumor cells (TCs) obtained from 1,259 HER2 [Ventana anti-HER2/neu (4B5) (Ventana Medical Systems, Tucson, AZ, US)] IHC-stained WSIs of breast cancer cases. The cell detection model may categorize cells into five classes: tumor cells with HER2 IHC 0 (H0); tumor cells with HER2 IHC 1+ (H1); tumor cells with HER2 IHC 2+ (H2); tumor cells with HER2 IHC 3+ (H3); and other cells (OT). H0 represents tumor cells (TCs) with no membrane staining, and H1 represents tumor cells (TCs) with incomplete membrane staining with faint or barely perceptible intensity. H2 represents tumor cells (TCs) with incomplete lateral or circumferential membrane staining with weak/moderate intensity. H3 represents tumor cells (TCs) with complete lateral or circumferential membrane staining with strong intensity.

The tissue segmentation model was trained using various datasets including 6.46×10² mm² of a cancer area (CA) from 1,214 PD-L1 22C3 [PD-L1 22C3 pharmDx IHC (Agilent Technologies Inc., Santa Clara, CA, US)] IHC-stained WSIs (including 197 colorectal cancer (CRC) cases), the various datasets consisting of biliary tract cancer, cancer, breast cancer, colorectal cancer, esophageal cancer, liver cancer, lung cancer, pancreatic cancer, prostate cancer, and stomach cancer.

The tissue segmentation model categorizes areas in WSI into a cancer area (CA) and a background (BG, which excludes CA). The performance of the tissue segmentation model was assessed using the intersection over union (IoU) metric. The evaluation was performed using a tuning dataset and an internal test dataset, and the tissue segmentation model achieved IoU values of 65.16 and 63.48 for CA and BG, respectively. In the case of the tissue detection model, the entire WSI was analyzed pixel by pixel, and the pixel corresponding to the tumor area was displayed.

By integrating information from the cell detection model and tissue segmentation model, only tumor cells (TCs) located within the cancer area (CA) were considered for HER2 evaluation. The proportion of tumor cells (TCs) exhibiting each staining intensity in the cancer area (CA) was calculated using Equation 1 below.

$$Al - Hn \cdot prop ( \% ) = \frac{{H}_{n}}{{H}_{0} + {H}_{1} + {H}_{2} + {H}_{3}} \times 100 \% \qquad [ Equation 1 ]$$

$$( where n = 0 , 1 , 2 , 3 )$$

Based on international consensus diagnostic criteria, the HER2 status was categorized using the AI-powered HER2 analyzer as HER2 IHC 3+ positive (AI-H3 prop>10%), HER2 IHC 2+ equivocal (AI-H3 prop<10% or AI-H2 prop>10%), HER2 IHC 1+ negative (AI-H1 prop>10%), and HER2 IHC 0 negative (AI-H1 prop≤10% or only H₀ cells are present).

### 2-2. Development of AI-Powered TME Analyzer

In an embodiment, an AI-power tumor microenvironment analyzer may be used to analyze the tumor microenvironment (TME).

The AI-powered tumor microenvironment analyzer according to an embodiment may be based on the DeepLabV3+ architecture. The AI-powered TME analyzer may include two deep learning (DL)-based AI models: a cell detection model; and a tissue segmentation model.

The AI-powered tumor microenvironment (TME) analyzer was trained using a dataset including 17,524 H&E-stained WSIs (tissue area: 14.6×10⁹ μm², TCs 2.2×10⁶), which were annotated by pathologists. To further enhance capabilities, the analyzer was updated with additional data for tumor cells (TCs), lymphocytes (LCs), macrophages (MPs), fibroblasts (FBs), endothelial cells (ECs), and other cells (OT) from various cancers, including colorectal cancer (CRC).

Specifically, the tissue segmentation model divides regions in WSI into a cancer area (CA), a cancer stroma (CS) area, and a background (BG), and by combining information derived from the cell detection model, cell density in each area for each cell type was calculated.

The density of tumor cells (TCs) in the cancer area (CA) and the cancer stroma (CS) area was defined as TC-CA and TC-CS (number of cells (counts/mm²). In addition, definitions were also made for lymphocytes, macrophages, fibroblasts, and endothelial cells by using the above-described method (LC-CA, LC-CS, MP-CA, MP-S, FB-CA, FB-CS, EC-CA, and EC-CS).

### Example 3. Analysis of HER2 Status in Colorectal Cancer Patients Through AI-Powered HER2 QCS Analysis

### 3-1. AI-Powered HER2 QCS Analysis

To analyze the HER2 status of colorectal cancer patients, the quantitative proportion (AI-H0 prop, AI-H1 prop, AI-H2 prop, and AI-H3 prop) of tumor cells (TCs) having each HER2 staining intensity (HER2 IHC 0 (H0), HER2 IHC 1+ (H1), HER2 IHC 2+ (H2), and HER2 IHC 3+ (H3)) was confirmed using the AI-powered HER2 analyzer described in 2-1 above.

Specifically, the HER2 IHC score (Negative, 1+, 2+, or 3+) on pathology slides from patients with colorectal cancer were determined in the Prescreening cohort (N=144) and the TRIUMPH cohort (N=30) by using the AI-powered HER2 analyzer of Example 2-1 in accordance with international consensus diagnostic criteria.

The proportions of HER2 amplification were confirmed through HER2 immunohistochemistry (IHC) and fluorescence in situ hybridization (FISH) or circulating tumor DNA (ctDNA) analysis in the TRIUMPH study.

Meanwhile, for pathology slides of each case in the Prescreening cohort (N=144) and the TRIUMPH cohort (N=30), tumor cells (TCs) were classified into tumor cells (TCs) having HER2 staining intensity: HER2 IHC 0 (H0); HER2 IHC 1+ (H1); HER2 IHC 2+ (H2); or HER2 IHC 3+ (H3), through AI-powered HER2 QCS analysis, and the proportion (AI-H0 prop, AI-H1 prop, AI-H2 prop, and AI-H3 prop) of tumor cells having each HER2 staining intensity was derived.

Specifically, cells were classified, by the AI-powered HER2 analyzer, as tumor cells having HER2 staining intensity: HER2 IHC 0 (H0); HER2 IHC 1+ (H1); HER2 IHC 2+ (H2); or HER2 IHC 3+ (H3) and other cells (OT). Tumor cells with no membrane staining were classified as tumor cells with the HER2 IHC 0 (H0) staining intensity, and tumor cells with incomplete membrane staining with faint or barely perceptible intensity were classified as tumor cells having the HER2 IHC 1+ (H1) staining intensity. Tumor cells with incomplete lateral or circumferential membrane staining with weak/moderate intensity were classified as tumor cells with the HER2 IHC 2+ (H2) staining intensity, and tumor cells with complete lateral or circumferential membrane staining with strong intensity were classified as tumor cells with the HER2 IHC 3+ (H3) staining intensity. The proportion (AI-H0 prop, AI-H1 prop, AI-H2 prop, and AI-H3 prop) of tumor cells having each HER2 staining intensity in pathology slide images was derived on the basis if the classification results. The results of analyzing HER2 status in the Prescreening cohort (N=144) and the TRIUMPH cohort through the AI-powered HER2 analyzer are illustrated in FIG. 5.

FIG. 5 illustrates the results of confirming the HER2 status of colorectal cancer patients as HER2 IHC scores and proportions of HER2 gene amplification of pathologic slides of the patients.

FIG. 5A illustrates the results of evaluating HER2 IHC scores in Prescreening cohort (n=144) and TRIUMPH cohort (n=30) by using an AI-powered HER2 quantification continuous score (QCS) analyzer, and FIG. 5B illustrates the results of analyzing proportions of HER2 amplification through the results of immunohistochemistry (IHC) and fluorescence in situ hybridization (FISH) or the results of circulating tumor DNA analysis (ctDNA) in the TRIUMPH study, in which the status of HER2 overexpression or gene amplification indicated as HER2 positive (HER2+).

As illustrated in FIG. 5A, the HER2 IHC scores in the Prescreening cohort (N=144) were as follows: 86 cases (59.7%) were HER2 IHC negative, 7 cases (4.9%) were analyzed as HER2 IHC 1+, 10 cases (6.9%) were analyzed as HER2 IHC 2+, and 41 cases (28.5%) were analyzed as HER2 IHC 3+. The HER2 IHC scores in the TRIUMPH cohort (N=30) were as follows: 3 cases (10%) were HER2 IHC negative, 2 cases (6.7%) were analyzed as HER2 IHC 1+, 2 cases (6.7%) were analyzed as HER2 IHC 2+, and 23 cases (76.7%) were analyzed as HER2 IHC 3+.

As illustrated in FIG. 5B, as result of analyzing HER2 IHC scores and FISH results in the TRIUMPH cohort, amplification was shown in 27 cases (90%), and only 3 cases (10%) was confirmed as negative (HER2). In addition, the ctDNA analysis also showed trends similar to the HER2 IHC scores and the FISH analysis results: 25 cases (83.3%) were HER2+ (positive), 4 cases (13.3%) were HER2-(negative), and 1 case (3.3%) remained unexamined.

Through the results shown in FIGS. 5A and 5B, it was confirmed that the results of evaluating HER2 IHC scores in the TRIUMPH cohort (n=30) by using the AI-powered HER2 analyzer showed trends similar to the HER2 IHC scores and FISH analysis results of the TRIUMPH cohort consisting of patients confirmed as HER2 positive through tissue and/or ctDNA analysis in the HER2-Screening and GOZILA studies. This means that the analysis results obtained by using the AI-powered HER2 QCS analyzer match the HER2 status of actual patients.

### 3-2. Analysis of HER2 Status in Colorectal Cancer Patients

The HER2 status of colorectal cancer patients enrolled in the TRIUMPH trial was confirmed through HER2 IHC, FISH, and ctDNA analysis results, and compared with the AI-powered HER2 QCS analysis results of Example 3-1.

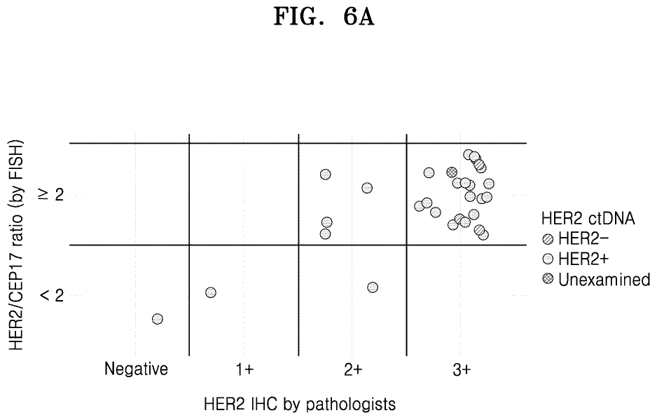

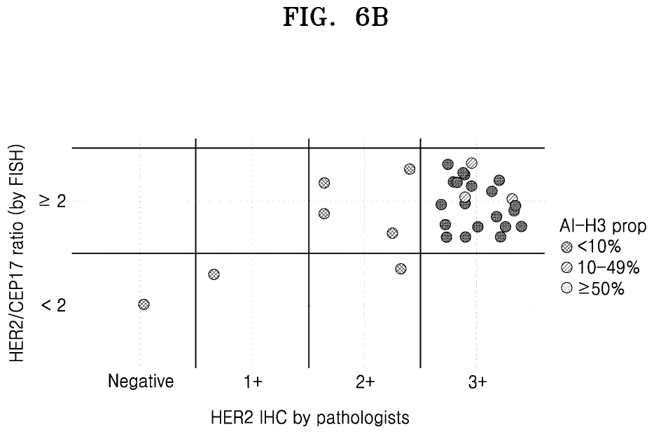

FIG. 6 is a graph showing the results of analyzing the HER2 status of patients enrolled in the TRIUMPH trial by using HER2 IHC, FISH and ctDNA analysis results and the proportion (AI-H3 prop, %) of tumor cells exhibiting HER2 IHC 3+ (H3) staining intensity determined by an AI model (the x-axis represents HER2 immunohistochemistry (IHC) scores evaluated by pathologists, and the y axis represents a HER2/CEP17 ratio determined through fluorescence in situ hybridization (FISH).

FIG. 6A illustrates HER2+ (positive), HER2-(negative), or an unexamined case, which are determined through HER2 ctDNA analysis, with colored dots, and FIG. 6B illustrates the proportion (AI-H3 prop, %) of tumor cells exhibiting HER2 IHC 3+ (H3) staining intensity, determined by the AI model, with colored dots.

As illustrated in FIG. 6, through HER2 IHC, FISH and ctDNA analysis results in the TRIUMPH study, it was confirmed that most of the patients analyzed as HER2 positive (HER2+) corresponds to the case in which the proportion (AI-H3 prop) of tumor cells having HER2 IHC 3+ (H3) staining intensity through AI-powered HER2 QCS analysis is 50% or more.

Meanwhile, the proportion (AI-H3 prop) of tumor cells having HER2 IHC 3+ (H3) staining intensity was evaluated through the AI-powered HER2 QCS analysis in the Prescreening cohort and the TRIUMPH cohort, and AI-H3 prop was shown for each HER2 IHC score of each pathology slide.

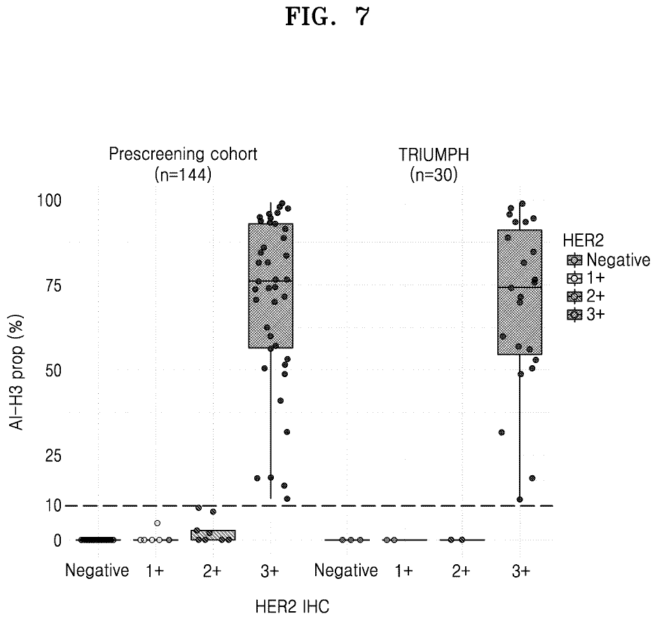

FIG. 7 is a graph showing the results of evaluating the proportion (AI-H3 prop) of tumor cells exhibiting HER2 IHC 3+ (H3) staining intensity in the respective pathology slides (WSI) of Prescreening cohort and TRIUMPH cohort by using an AI-powered HER2 QCS analyzer.

As illustrated in FIG. 7, in the Prescreening cohort, the median AI-H3 prop was 0% for HER2 IHC negative, HER2 IHC 1+, and HER2 IHC 2+, with a range from 0.00% to 0.39% (min-max), 0.00% to 4.87%, and 0.00% to 9.43%, respectively. However, for the HER2 IHC 3+ cases, a high median of 75.8% (min-max 11.9% to 99.0%) was observed. The cases in the TRIUMPH cohort were confirmed to show patterns similar to the Prescreening cohort, with median AI-H3 prop of 0% (min-max 0.00% to 0.0003%), 0% (0.00% to 0.00%), 0.005% (0.00% to 0.009%), and 73.9% (11.9% to 99.0%), respectively, for HER2 IHC negative, HER2 IHC 1+, HER2 IHC 2+, and HER2 IHC 3+.

Meanwhile, it was confirmed that the proportion of tumor cells (AI-H3 prop) with HER2 IHC 3+ (H3) staining intensity in HER2 IHC 3+ cases showed considerable variability. Specifically, a low AI-H3 prop was 18.1% and a high AI-H3 prop was 97.4%. Cases corresponding to the ranges of low AI-H3 prop and high AI-H3 prop evaluated through the AI-powered HER2 QCS analyzer were confirmed through representative clinical cases from the TRIUMPH study.

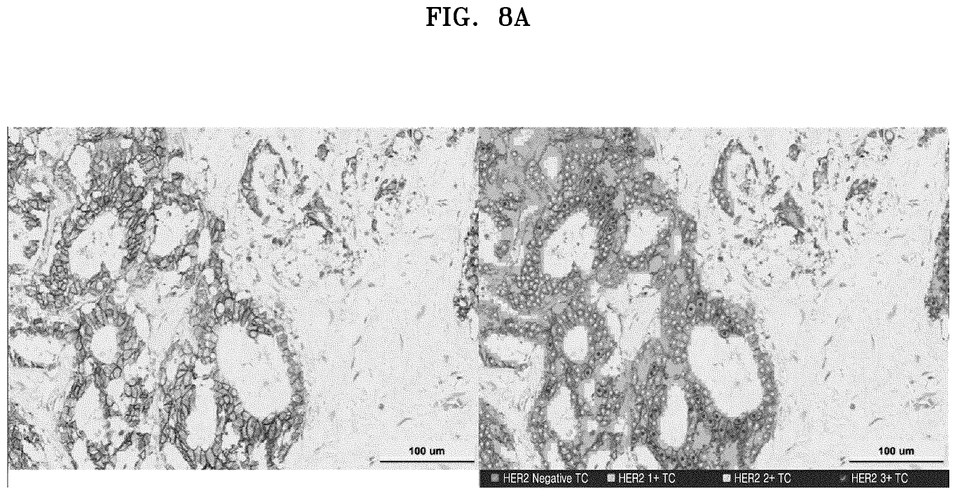

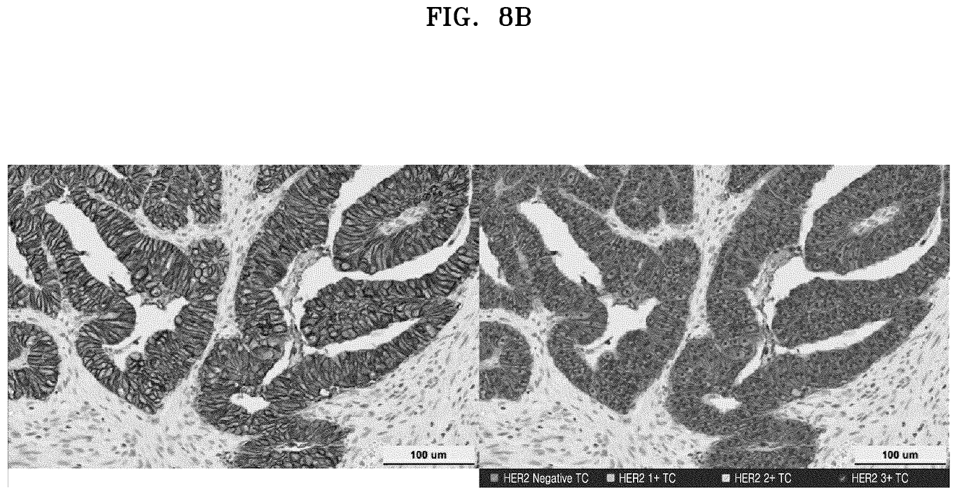

FIG. 8 illustrates images showing representative clinical cases of TRIUMPH cohort with low (18.1%) AI-H3 prop and high (97.4%) AI-H3 prop in pathology slides with HER2 IHC 3+ score.

FIG. 8A is an image showing a representative clinical case with low (18.1%) AI-H3 prop in the pathology slide with HER2 IHC 3+ score in the TRIUMPH cohort, and FIG. 8B is an image showing a representative clinical case having high (97.4%) AI-H3 prop in the pathology slide with HER2 IHC 3+ score in the TRIUMPH cohort.

FIGS. 8A and 8B are both clinical cases classified as slides with a HER2 IHC 3+ score by the AI-powered HER2 QCS analyzer, wherein the left image is an original WSI, and the right image is a WSI processed by the AI analyzer. (Tumor cells (TCs) are color-coded according to HER2 staining intensity (Blue: HER2 negative (HER2 IHC 0 (H0)) TC, Green: HER2 1+ (HER2 IHC 1+ (H1)) TC, Yellow: HER2 2+ (HER2 IHC 2+ (H2)) TC, Red: HER2 3+ (HER2 IHC 3+ (H3)) TC), and the green-shaded area indicates a cancer area.

As illustrated in FIGS. 7 and 8, it was confirmed that the proportion (AI-H3 prop) of tumor cells having HER2 IHC 3+ (H3) staining intensity in pathology slides with a HER2 IHC 3+ score showed considerable variability, and these results were also confirmed in actual clinical outcomes. This means that, even when the pathology slides of patients are determined to have a HER2 IHC 3+ score, the proportions of tumor cells having HER2 IHC 3+ (H3) staining intensity in the pathology slides are distributed in various ways.

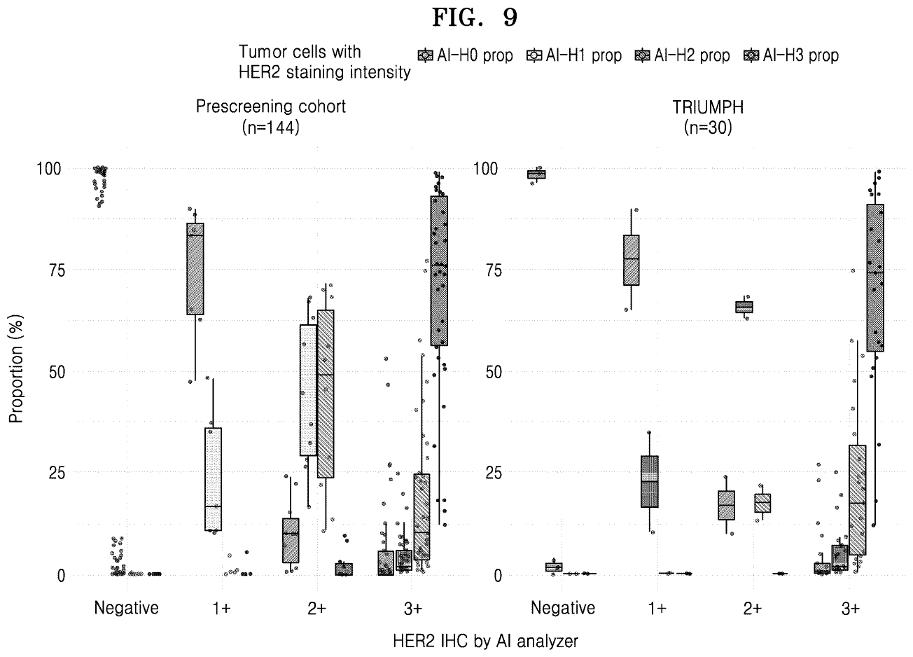

More specifically, the results of evaluating the proportions of tumor cells (TCs) having staining intensities (HER2 IHC 0 (H0), HER2 IHC 1+ (H1), and HER2 IHC 2+ (H2)) other than the HER2 IHC 3+ (H3) staining intensity, as classified by the AI-powered HER2 QCS analyzer in the Prescreening cohort and the TRIUMPH cohort are illustrated in FIG. 9.

FIG. 9 is a graph showing the proportions of tumor cells (TCs) with HER2 staining intensities of HER2 IHC 0 (H0), HER2 IHC 1+ (H1), HER2 IHC 2+ (H2) and HER2 IHC 3+ (H3) in slides according to HER2 IHC scores classified by an AI-powered HER2 QCS analyzer in the Prescreening cohort and the TRIUMPH cohort. In FIG. 9, the x-axis represents the HER2 IHC score of the pathology slides determined by the AI-powered HER2 analyzer, and the proportion (negative, 1+, 2+, and 3+ are represented as AI-H0 prop, AI-H1 prop, AI-H2 prop, and AI-H3 prop) of tumor cells exhibiting each HER2 staining intensity (HER2 IHC 0 (H0), HER2 IHC 1+ (H1), HER2 IHC 2+ (H2), or HER2 IHC 3+ (H3)) in a group having each HER2 IHC score was plotted as a box plot by color.

As illustrated in FIG. 9, it was confirmed that the proportion (AI-H3 prop) of tumor cells with HER2 IHC 3+ (H3) staining intensity was significantly higher in pathology slides with a HER2 IHC 3+ score.

### Example 4. Confirmation of Agreement Between HER2 IHC Scores Evaluated by AI-Powered HER2 Analyzer and Pathologists

To confirm agreement between HER2 IHC scores of pathology slide images acquired from tissues of patients, as evaluated by the AI-powered HER2 analyzer and pathologists, the agreement was calculated.

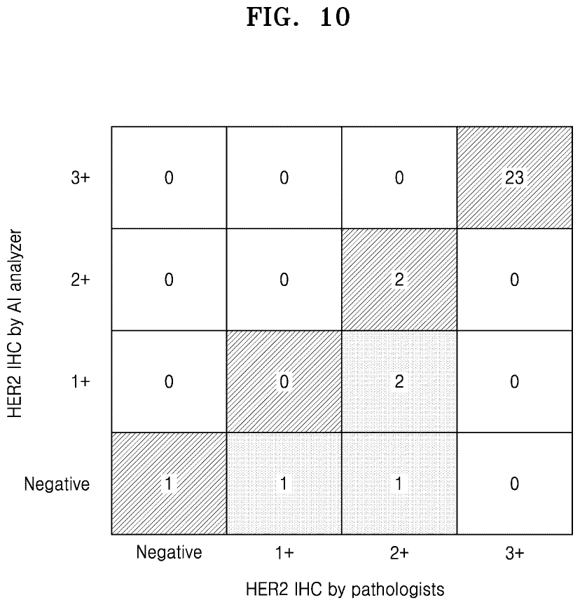

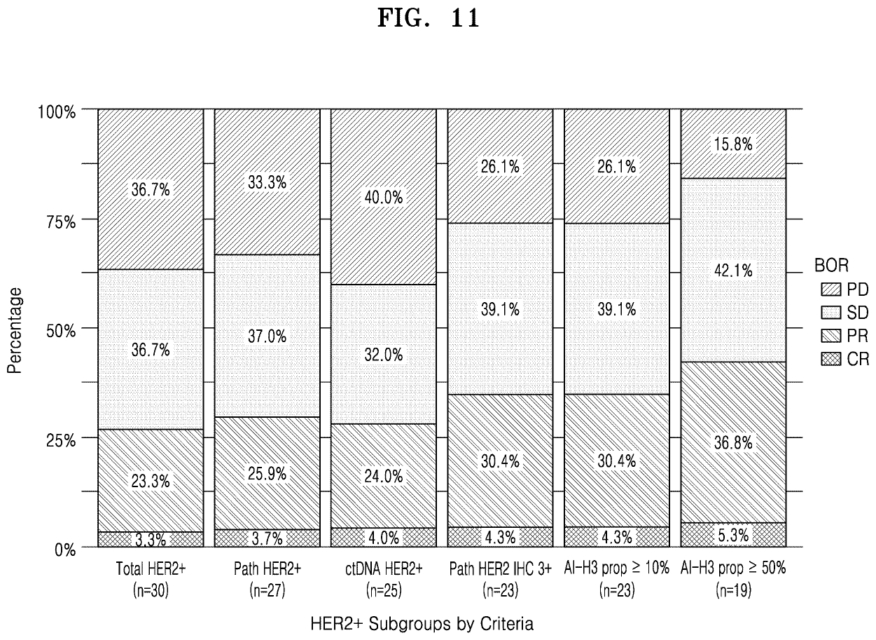

Specifically, the x-axis represents the HER2 IHC score of pathology slides as evaluated by pathologists, and the y-axis represents the HER2 IHC score of pathology slides as determined by the AI-powered HER2 analyzer, and the agreement therebetween was calculated. The agreement was calculated in a horizontal direction and indicated by blue gradation, and the results thereof are illustrated in FIG. 10.

FIG. 10 illustrates the results of confirming the agreement between HER2 IHC scores of pathology slides, evaluated for TRIUMPH cohort patients by an AI-powered HER2 QCS analyzer and pathologists.

As illustrated in FIG. 10, the agreement between the HER2 IHC scores of pathology slides as determined by the AI-powered HER2 QCS analyzer and the HER2 IHC scores of pathology slides as evaluated by pathologists was confirmed to be 86.7% (95% confidence intervals [95% CI] 69.3% to 96.2%, Cohen's kappa 0.663). In particular, the accuracy for pathology slides with a HER2 IHC 3+ score was 100%.

### Example 5. Clinical Outcome Analysis of HER2-Targeted Therapeutic Agent By Using AI-Powered HER2 QCS Analysis

### 5-1. Clinical Outcome Analysis Through Tumor Response Rate

The clinical outcomes of patients in the TRIUMPH cohort (n=30) who were administered with pertuzumab and trastuzumab (dual HER2-targeted therapy) were analyzed. Specifically, an objective response rate (ORR) was investigated to confirm the effect of pertuzumab and trastuzumab administration.

A tumor response rate was evaluated based on the Response Evaluation Criteria in Solid Tumors (RECIST) version 1.1. While responses for each case were determined by local site investigators and through independent central review, this study exclusively utilized only the assessments conducted by local site investigators.

For the tumor response rate, it was compared whether there was a difference in objective response rate (ORR) between the following subgroups. In this embodiment, the ORR refers to the proportion of patients who achieved a complete response (CR) or partial response (PR) confirmed in a follow-up test for more than 4 weeks after the initial response.

TABLE 2

| Subgroup | Classification criteria |
|---|---|
| Total HER2+ | HER2+ (positive) confirmed by pathologist- |
| | assessed HER2 IHC with FISH or ctDNA |
| Path HER2+ | HER2 IHC 3+ confirmed by pathologist-assessed |
| | HER2 IHC or HER2 IHC 2+ with FISH+ |
| ctDNA HER2+ | HER2+ (positive) confirmed by ctDNA |
| Path HER2 IHC 3+ | HER2 IHC 3+ confirmed by pathologist- |
| | assessed HER2 IHC |
| Al—H3 prop ≥ 10% | Proportion of tumor cells with HER2 IHC |
| | 3+ (H3) staining intensity ≥ 10% |
| Al—H3 prop ≥ 50% | Proportion of tumor cells with HER2 IHC |
| | 3+ (H3) staining intensity ≥ 50% |

Clinical outcomes (CR, complete response; PR, partial response; SD, stable disease; PD, progressive disease) for analyzing the objective response rate (ORR) were confirmed through the best overall response (BOR) for each subgroup as shown in Table 2, and the results thereof are illustrated in FIG. 11.

FIG. 11 is a graph showing the clinical outcomes of groups with total HER2+, Path HER2+, ctDNA HER2+, Path HER2 IHC 3+, AI-H3 prop≥10%, and AI-H3 prop≥50%.

As illustrated in FIG. 11, the ORR of TRIUMPH cohort patients (total HER2+) administered with pertuzumab and trastuzumab was found to be 26.7% ( 8/30). This is consistent with the results showing ORRs of 29.6% (8/27) and 28.0% (7/25), respectively, for cases (Path HER2+, ctDNA HER2+) evaluated as HER2+ (positive) by pathologist-assessed HER2 IHC with FISH and by ctDNA.

In addition, the agreement between groups classified as HER2 IHC 3+ by pathologists and groups determined as AI-H3 prop≥10% was 100%, resulting in identical ORRs of 34.7% (8/23) for both groups. In particular, it was confirmed that, when the cutoff was set at AI-H3 prop≥50%, the ORR increased to 42.1% (8/19).

### 5-2. Clinical Outcome Analysis Through Tumor Regression Percentages

The clinical outcomes of patients in the TRIUMPH cohort (n=30) who were administered with pertuzumab and trastuzumab (dual HER2-targeted therapy) were analyzed.

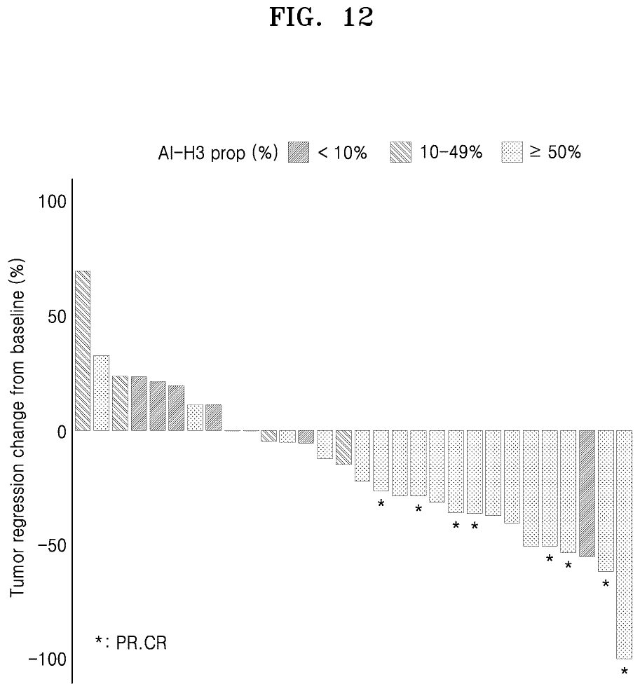

Specifically, to confirm the effect of pertuzumab and trastuzumab administration, tumor regression percentages were confirmed, and the results are shown in FIG. 12.

FIG. 12 is a waterfall plot showing tumor regression percentages from the baseline of TRIUMPH trial patients administered with pertuzumab and trastuzumab.

As illustrated in FIG. 12, among the cases exhibiting a reduction in tumor size from baseline, 77.3% (17/22) satisfied AI-H3 prop≥50%, 9.1% (2/22) had an AI-H3 prop between 10% and 49%, and 13.6% (3/22) satisfied AI-H3 prop<10%.

This indicates that, in patients who were administered with pertuzumab and trastuzumab and confirmed as HER2 positive (overexpression or gene amplification), the case of AI-H3 prop≥10% means that treatment prognosis according to the administration of pertuzumab and trastuzumab is highly likely to be good (86.4%), and in particular, the case of AI-H3 prop≥50% means that treatment prognosis according to the administration of pertuzumab and trastuzumab is very highly likely to be good (the rate of reduction in tumor size from baseline is high) (77.3%).

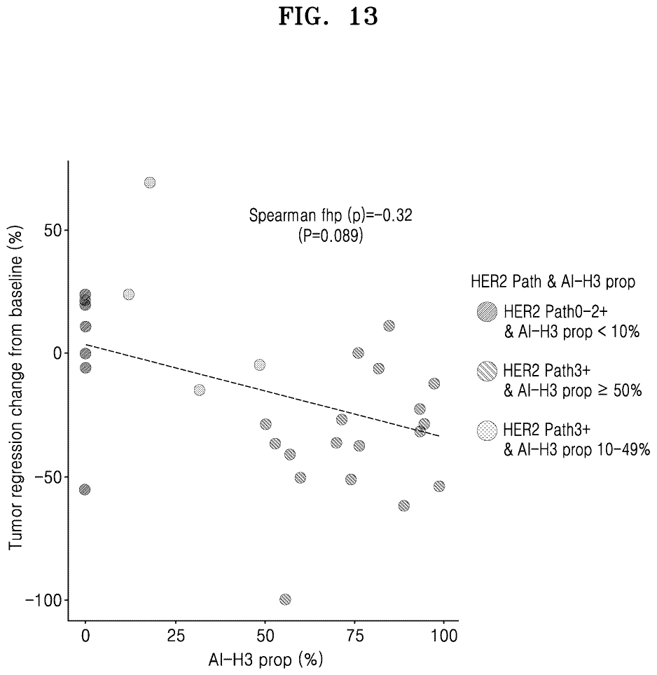

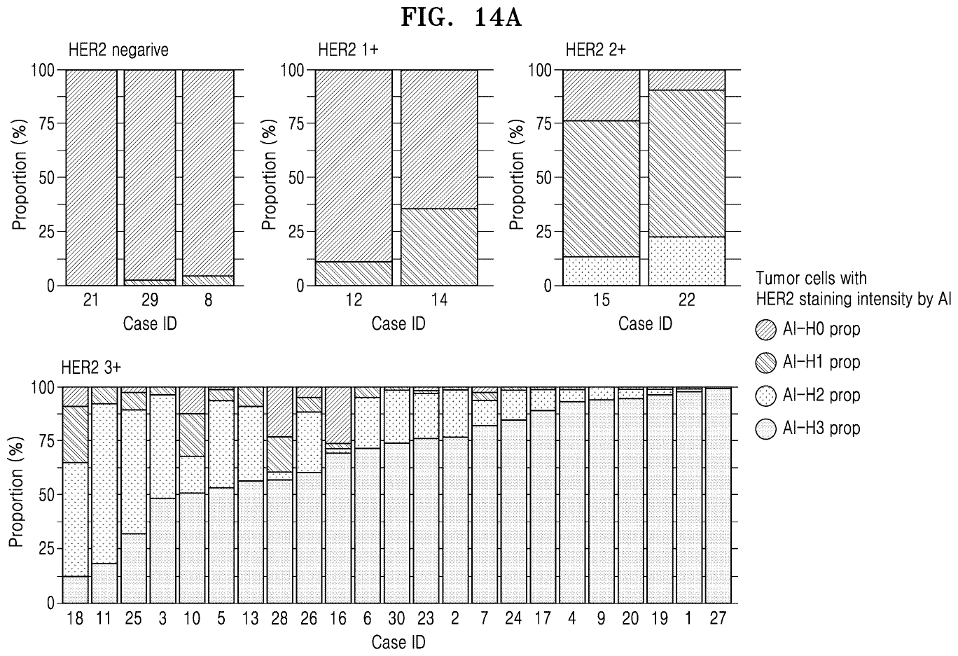

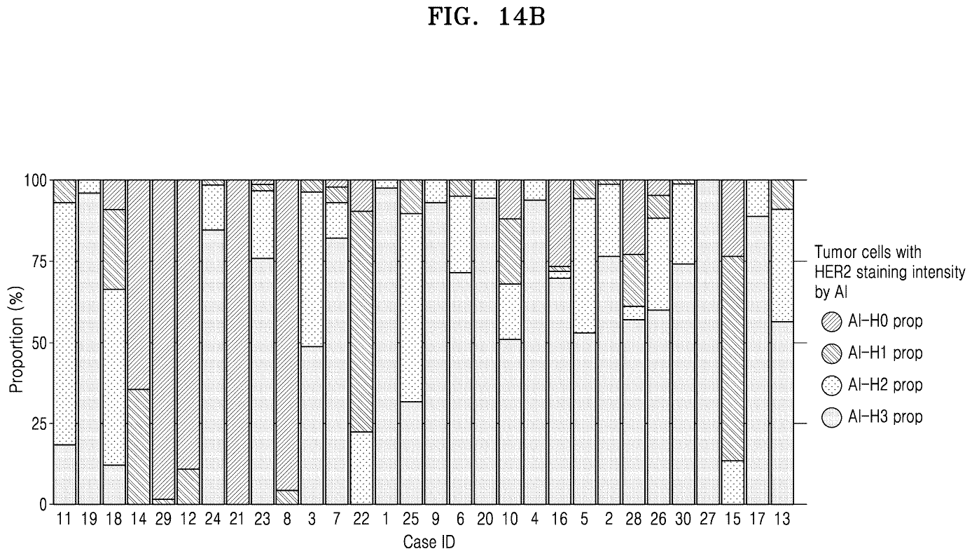

Meanwhile, the correlation between the proportion (AI-H3 prop) of tumor cells exhibiting HER2 IHC 3+ (H3) staining intensity as measured by the AI-powered HER2 analyzer and tumor regression changes of TRIUMPH trial patients administered with pertuzumab and trastuzumab was analyzed through Spearman's correlation analysis, and the results thereof are illustrated in FIG. 13.

FIG. 13 is a graph showing the correlation between the proportion of tumor cells exhibiting HER2 IHC 3+ (H3) staining intensity, measured by an AI-powered HER2 analyzer, and tumor regression changes of TRIUMPH trial patients administered with pertuzumab and trastuzumab. In FIG. 13, the y-axis represents tumor regression percentages compared to baseline of TRIUMPH patients administered with pertuzumab and trastuzumab, the x-axis represents AI-H3 prop (%), and the dots in each color represent combined results of the proportion (AI-H3 prop, %) of tumor cells exhibiting HER2 IHC 3+ (H3) staining intensity, measured by the AI-powered HER2 analyzer, and HER2 IHC scores (HER2 Path) evaluated by pathologists.

As illustrated in FIG. 13, as a result of Spearman's correlation analysis, Spearman's rho (p) was found to be −0.32 (p=0.089), and a borderline negative correlation between tumor regression changes and AI-H3 prop was confirmed.

This means that, when AI-H3 prop is 50% or more, drug sensitivity and treatment prognosis for pertuzumab and trastuzumab are likely to be good.

In addition, whole slide images (WSIs) in TRIUMPH cohort cases were classified as HER2 IHC scores by using the AI-powered HER2 analyzer, and the proportions (represented as AI-H0 prop, AI-H1 prop, AI-H2 prop, and AI-H3 prop, respectively) of tumor cells having staining intensity levels of HER2 IHC 0 (H0), HER2 IHC 1+ (H1), HER2 IHC 2+ (H2), and HER2 IHC 3+ (H3) in each case were measured, and the results thereof are illustrated in FIG. 14.

FIG. 14 is a set of graphs showing the proportion (AI-H0 prop, AI-H1 prop, AI-H2 prop or AI-H3 prop) of tumor cells having each HER2 staining intensity (HER2 IHC 0 (H0), HER2 IHC 1+ (H1), HER2 IHC 2+ (H2), or HER2 IHC 3+ (H3)) in the TRIUMPH study.

FIG. 14A is a set of graphs showing the distribution of proportions (AI-H0 prop, AI-H1 prop, AI-H2 prop, and AI-H3 prop) of tumor cells having HER2 staining intensity of HER2 IHC 0 (H0), HER2 IHC 1+ (H1), HER2 IHC 2+ (H2) or HER2 IHC 3+ (H3) in TRIUMPH cohort slides, in which the slides were classified as HER2 IHC scores, and FIG. 14B is a graph showing the proportions (AI-H0 prop, AI-H1 prop, AI-H2 prop and AI-H3 prop) of tumor cells having HER2 staining intensities of HER2 IHC 0 (H0), HER2 IHC 1+ (H1), HER2 IHC 2+ (H2), and HER2 IHC 3+ (H3) for each TRIUMPH cohort case.

As illustrated in FIG. 14, it was confirmed that the proportions of tumor cells having HER2 staining intensities: HER2 IHC 0 (H0), HER2 IHC 1+ (H1), HER2 IHC 2+ (H2), and HER2 IHC 3+ (H3) were different according to the HER2 IHC scores of the TRIUMPH cohort slides, and the proportions (AI-H0 prop, AI-H1 prop, AI-H2 prop, and AI-H3 prop) of tumor cells having HER2 staining intensities: HER2 IHC 0 (H0), HER2 IHC 1+ (H1), HER2 IHC 2+ (H2), and HER2 IHC 3+ (H3) were different for each TRIUMPH cohort case.

Through the results shown in FIGS. 12 to 14, it was confirmed that AI-H3 prop was different depending on patients, and when the HER2 IHC score for slides of patients is HER2 IHC 3+, the proportion (AI-H3 prop) of tumor cells having HER2 IHC 3+ (H3) staining intensity was high.

### 5-3. Clinical Outcome Analysis Through Progression-Free Survival (PFS) and Overall Survival (OS)

The clinical outcomes of patients in the TRIUMPH cohort (n=30) who were administered with pertuzumab and trastuzumab (dual HER2-targeted therapy) were analyzed.

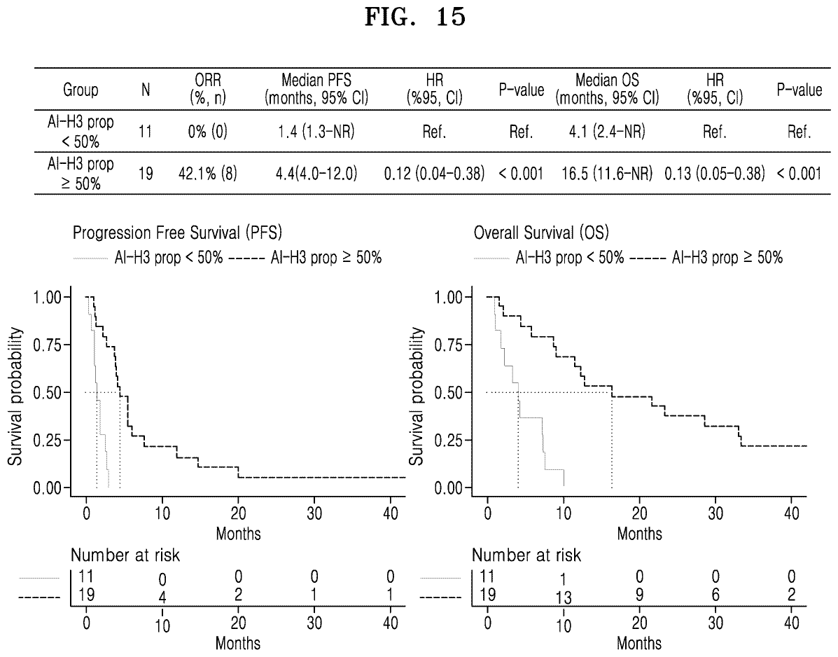

Specifically, the results of AI-powered HER2 QCS analysis were compared with progression-free survival (PFS) and overall survival (OS) according to the proportions (AI-H3 prop<50% and AI-H3 prop≥50%) of tumor cells exhibiting HER2 IHC 3+ (H3) staining intensity by using Kaplan-Meier curves and a Cox proportional hazard model, and the results thereof are illustrated in FIG. 15. FIG. 15 illustrates the overall response rates (ORRs) of progression-free survival (PFS) and overall survival (OS) and Kaplan-Meier curves, according to AI-H3 prop<50% or AI-H3 prop≥50%.

As illustrated in FIG. 15, the progression-free survival (PFS) was 1.4 months (95% confidence intervals, 1.3—not reached) for the group in which the proportion of tumor cells exhibiting HER2 IHC 3+ (H3) staining intensity as determined through AI-powered HER2 QCS analysis is 50% or more (AI-H3 prop<50%), and extended to 4.4 months (95% confidence intervals, 4.0-12.0) for the group with AI-H3 prop≥50%), and a hazard ratio (HR) was 0.12 (95% confidence intervals, 0.04-0.38; p<0.001). This means that the group with AI-H3 prop≥50% exhibited significantly better progression-free survival (PFS). Similarly, the overall survival (OS) was 4.1 months (95% confidence intervals, 2.4—not reached) in the group with AI-H3 prop<50%), and increased to 16.5 months (95% confidence intervals, 11.6—not reached) in the group with AI-H3 prop≥50%. This means that the group with AI-H3 prop≥50% exhibited significantly better overall survival (OS).

Thus, it was confirmed that, in the group where the proportion of tumor cells exhibiting HER2 IHC 3+ (H3) staining intensity as determined through AI-powered HER2 QCS analysis was 50% or more (AI-H3 prop≥50%), therapeutic responsiveness, drug sensitivity and treatment prognosis for pertuzumab and trastuzumab were highly likely to be good.

### Example 6. Clinical Outcome Analysis of HER2-Targeted Therapy Using AI-Powered Tumor Microenvironment Factor (TME) Analysis

### 6-1. AI-Powered Tumor Microenvironment Factor Analysis in Responders and Non-responders

To determine trends in tumor microenvironment (TME) factors, responders (patients who achieved complete response or partial response) and non-responders (patients who were able to achieve complete response or partial response) were compared for the entire TRIUMPH cohort. The analyzed tumor microenvironment (TME) factors were lymphocyte density (LC-CA and LC-CS), fibroblast density (FB-CA and FB-CS), macrophage density (MP-CA and MP-CS), and endothelial cell (EC) density (EC-CA and EC-CS), as analyzed in a cancer area (CA) and a cancer stroma (CS) area. The p-value for comparison in each factor was illustrated at the top of the box plot, and was calculated using the Student's t-test or Wilcoxon test.

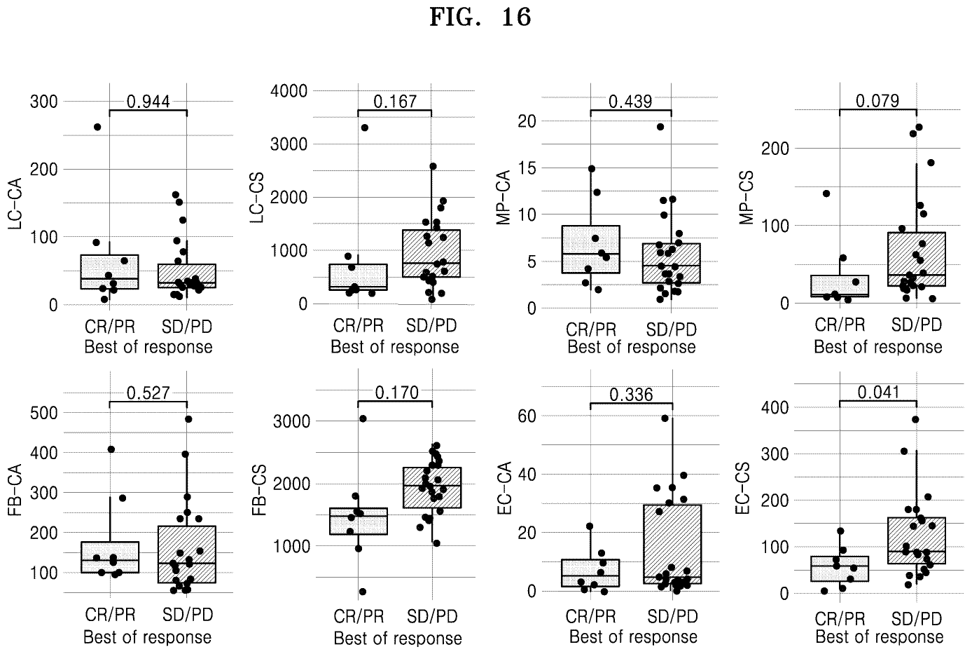

Specifically, the best overall response (BOR) was classified as complete response (CR), partial response (PR), stable disease (SD), or progressive disease (PD) in accordance with RECIST V1.1 criteria. Patients who achieved complete response (CR) or partial response (PR) were classified as responders, and patients who achieved stable disease (SD) or progressive disease (PR) were classified as non-responders. Comparative analysis of tumor microenvironment (TME) factors was performed in responders and non-responders classified according to the above criteria, and the results thereof are illustrated in FIG. 16.

FIG. 16 is a set of graphs showing the results of comparative analysis of AI-powered tumor microenvironment factors (lymphocyte (LC) density, macrophage (MP) density, fibroblast (FB) density, and endothelial cell (EC) density) between responders and non-responders of TRIUMPH patients.

As illustrated in FIG. 16, a tendency towards increased lymphocyte (LC), macrophage (MP), fibroblast (FB) and endothelial cell (EC) intensities in the cancer area (CA) was found in responders (referred to as LC-CA, MP-CA, FB-CA, and EC-CA, respectively).

In contrast, LC, MP and FB densities in the cancer stroma (CS) area (referred to as LC-CS, MP-CS, and FB-CS, respectively) were generally lower in responders, but these results had no statistical significance. The EC density in the cancer stroma (CS) area was confirmed to be significantly lower in responders than in non-responders, which is a significant result (median values of 56.8% [95% confidence intervals, 21.0-81.8] versus 89.3 [95% confidence intervals, 58.4-162.9], p=0.041).

This means that EC-CS can be used to select patients who have high drug sensitivity and good treatment prognosis for pertuzumab and trastuzumab.

### 6-2. Analysis of AI-Powered Tumor Microenvironment Factors According to AI-H3 Prop

The tumor microenvironment (TME) factors analyzed by AI were compared in the group with AI-H3 prop<50% and the group with AI-H3 prop≥50%, as determined by an AI-powered HER2 quantification continuous score (QCS) analyzer.

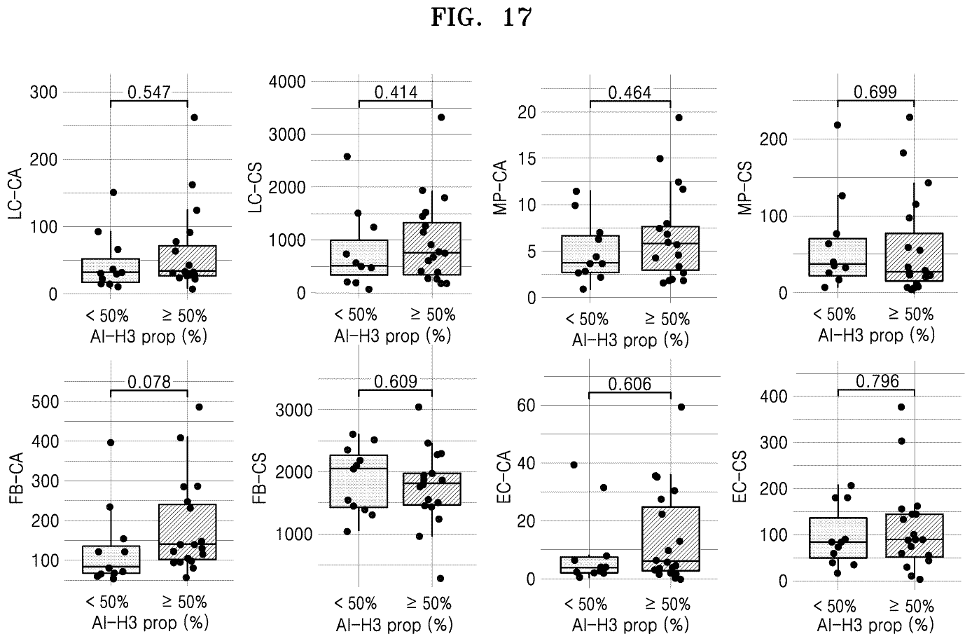

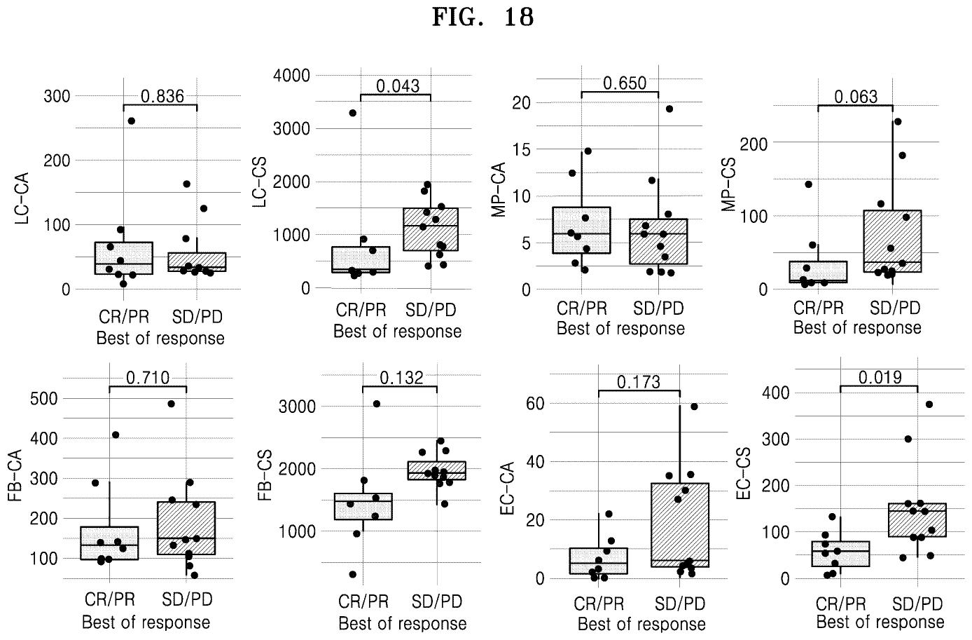

Specifically, the p-value for comparison in each factor was illustrated at the top of the box plot, and was calculated using the Student's t-test or Wilcoxon test. Lymphocyte density (LC-CA and LC-CS), fibroblast density (FB-CA and FB-CS), macrophage density (MP-CA and MP-CS) and endothelial cell density (EC-CA and EC-CS) in the cancer area (CA) and the cancer stroma (CS) area were analyzed, and the results thereof are illustrated in FIG. 17.

FIG. 17 is a set of graphs showing the results of comparison in each TME factor between a group with AI-H3 prop<50% and a group with AI-H3 prop≥50% in the TRIUMPH cohort.

As illustrated in FIG. 17, it was confirmed that there was no significant difference in any of the tumor microenvironment (TME) factors between the group with AI-H3 prop<50% and the group with AI-H3 prop≥50%.

This means that it may be difficult to select patients who can show high drug sensitivity and good treatment prognosis for pertuzumab and trastuzumab by using only each TME factor.

### 6-3. Analysis of AI-Powered Tumor Microenvironment Factors in Patients with AI-H3 Prop of 50% or More Classified as Responders and Non-Responders

The clinical outcomes of patients in the TRIUMPH cohort (n=30) who were administered with pertuzumab and trastuzumab (dual HER2-targeted therapy) were analyzed.

Specifically, in the TRIUMPH cohort, for patients with AI-H3 Prop≥50%, tumor microenvironment factors (TME factors) quantified using an AI-powered tumor microenvironment analyzer were compared between responders (patients who achieved complete response (CR) or partial response (PR) in accordance with RECIST V1.1 criteria) and non-responders (patients who achieved stable disease (SD) or progressive disease (PD)).

The analyzed tumor microenvironment (TME) factors were lymphocyte density (LC-CA and LC-CS), fibroblast density (FB-CA and FB-CS), macrophage density (MP-CA and MP-CS) and endothelial cell density (EC-CA and EC-CS) in the cancer area (CA) and the cancer stroma (CS) area, and the results of analysis of tumor microenvironment factors between responders and non-responders are illustrated in FIG. 18. The p-value for comparison in each tumor microenvironment factor was illustrated at the top of the box plot, and was calculated using the Student's t-test or Wilcoxon test.

FIG. 18 is a set of graphs showing the results of comparison in TME factors between responders (patients who achieved complete response (CR) or partial response (PR) according to RECIST V1.1 criteria) and non-responders (patients who developed stable disease (SD) or progressive disease (PD), the TME factors being quantified by an AI-powered TME analyzer in a group with AI-H3 prop≥50% of the TRIUMPH cohort.

As illustrated in FIG. 18, it was confirmed that LC-CA, MP-CA, FB-CA, and EC-CA were similar in both the responders and the non-responders, whereas LC-CS, MP-CS, FB-CS, and EC-CS were low in the responders. However, this trend was statistically significant only for LC-CS and EC-CS.

Specifically, median values of LC-CS between responders and non-responders were 297.1 (95% confidence intervals, 235.6-797.9) versus 1143.8 (95% confidence intervals, 686.7-1473.1), and p=0.043. It was confirmed that median values of EC-CS between responders and non-responders were 56.8 (95% confidence intervals, 21.0-81.8) versus 143.9 (95% confidence intervals, 87.9-159.6).

This means that patients with AI-H3 Prop≥50% and low LC-CS and EC-CS factors are highly likely to show high therapeutic responsiveness and drug sensitivity and good treatment prognosis for pertuzumab and trastuzumab.

### Example 7. Analysis of Progression-Free Survival (PFS) or Overall Survival (OS) According to Quantitative Proportion (AI-H3 Prop) of Tumor Cells Exhibiting AI-Powered HER2 IHC 3+ (H3) Staining Intensity and AI-Powered Tumor Microenvironment (TME) Factors

Progression-free survival (PFS) or overall survival (OS) according to the quantitative proportion (AI-H3 prop) of tumor cells exhibiting AI-powered HER2 IHC 3+ (H3) staining intensity and AI-powered tumor microenvironment factors were analyzed using Kaplan-Meier curves.

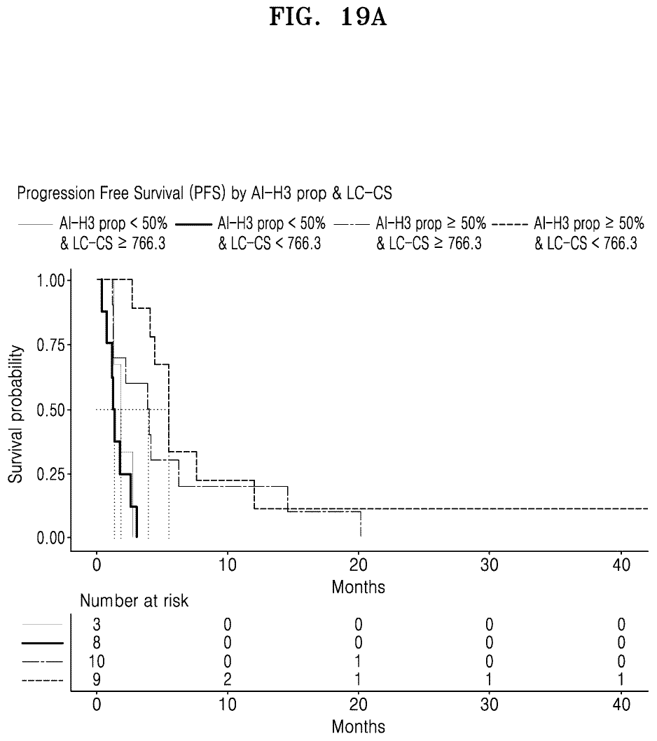

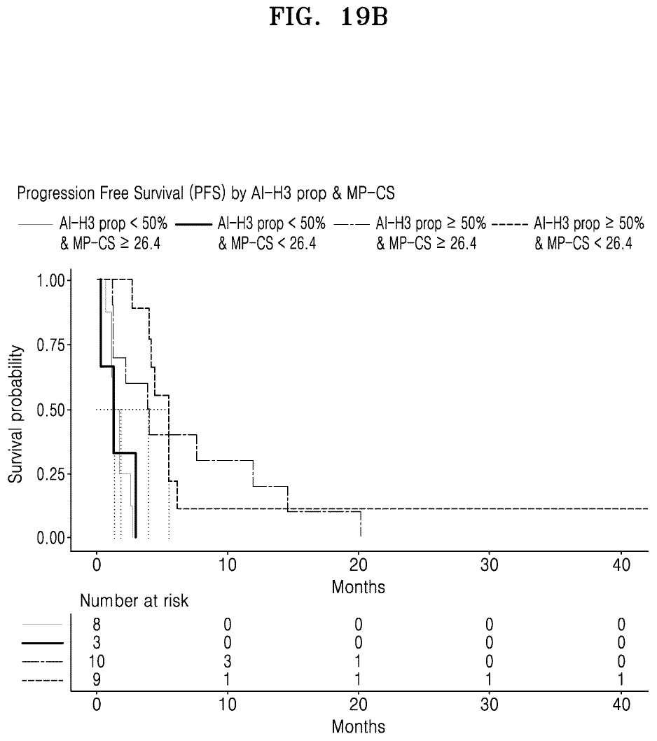

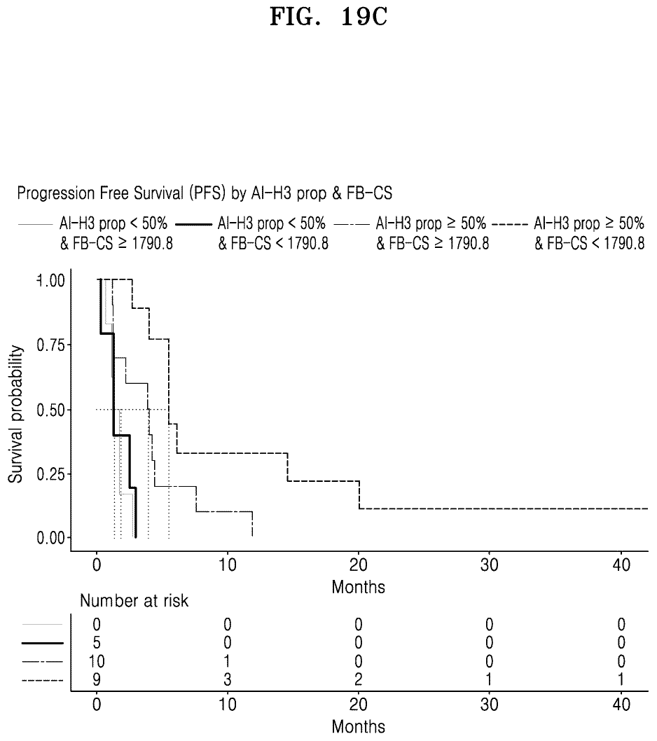

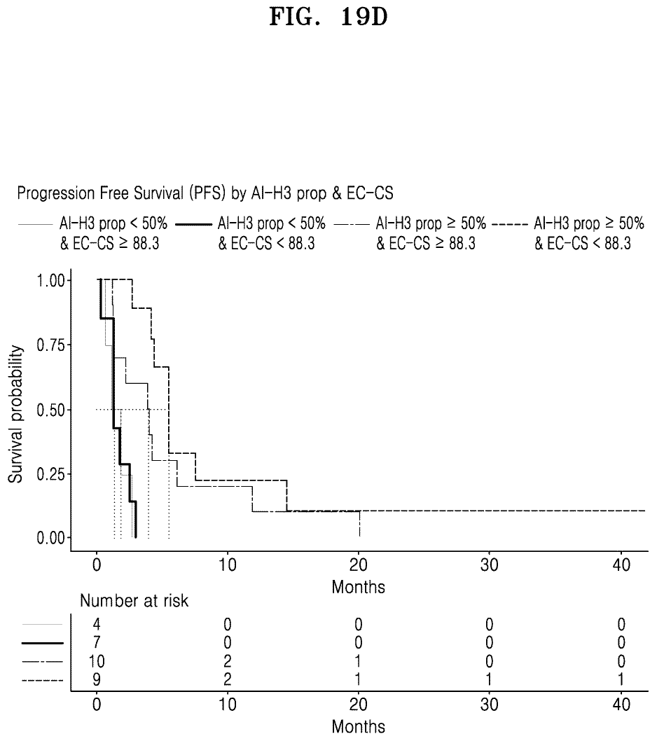

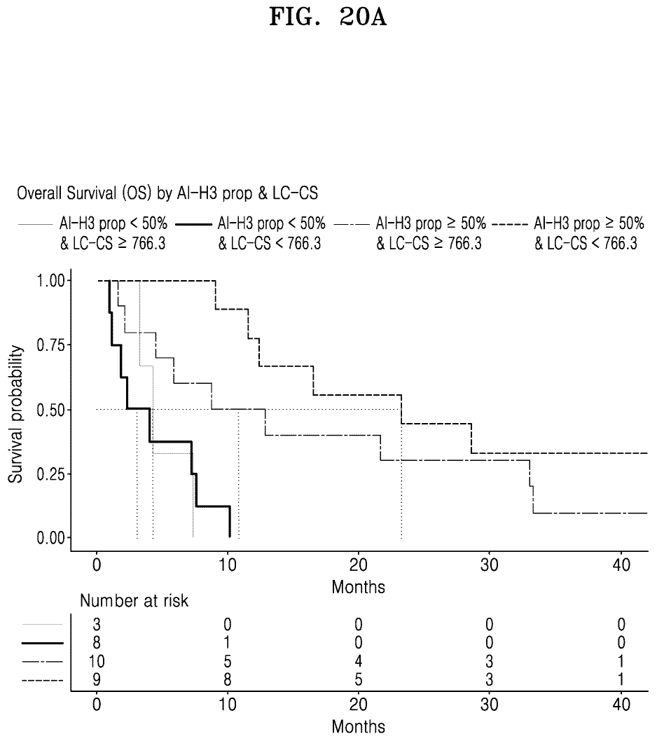

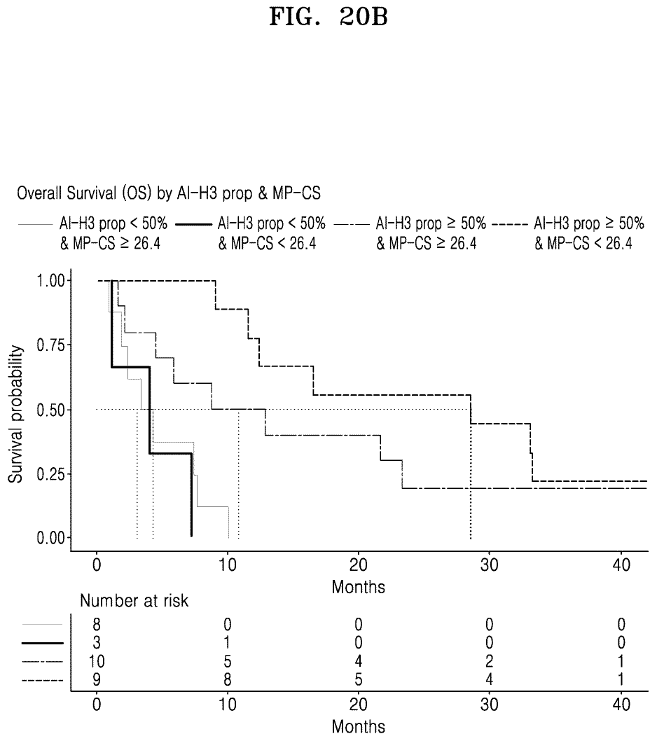

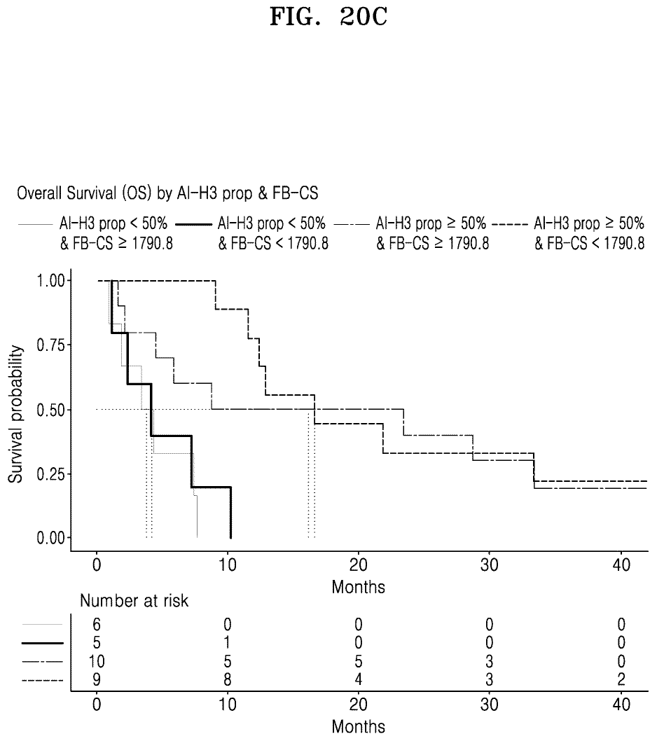

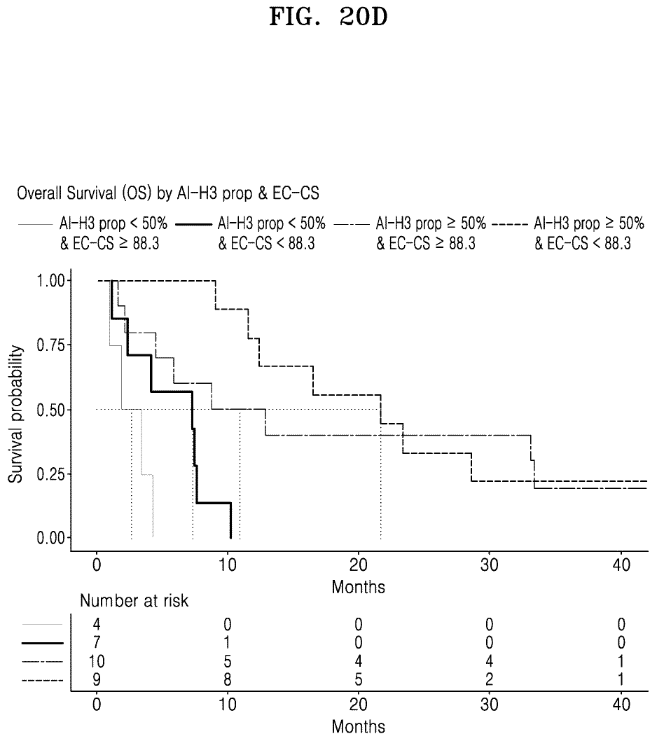

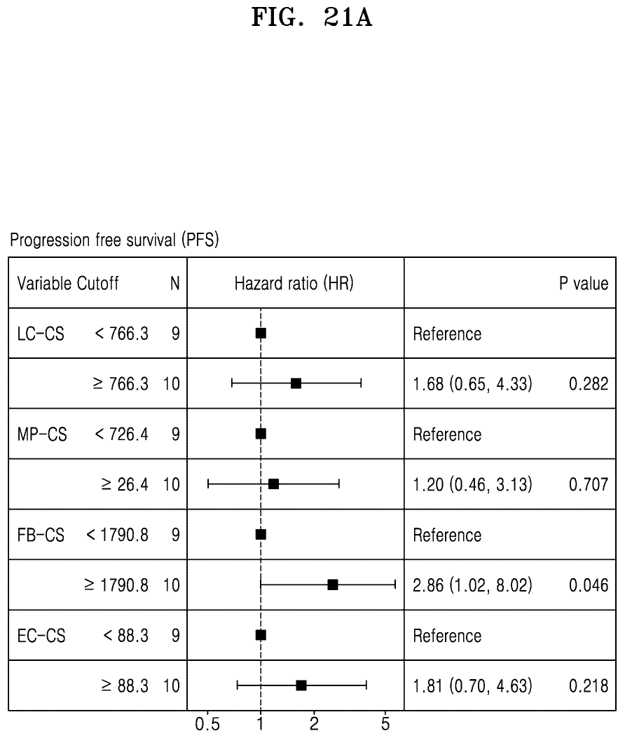

Specifically, 30 patients of the TRIUMPH cohort was divided into a group in which the proportion of tumor cells exhibiting HER2 IHC 3+ (H3) staining intensity is 50% or more (AI-H3 prop≥50%) or a group in which the proportion of tumor cells exhibiting HER2 IHC 3+ (H3) staining intensity is less than 50% (AI-H3 prop<50%), and progression-free survival (PFS) or overall survival (OS) was analyzed using Kaplan-Meier curves according to tumor microenvironment (TME) factors analyzed by AI. The Kaplan-Meier curves of a group in which AI-H3 prop and any one of the AI-analyzed tumor microenvironment factors (LC-CS, MP-CS, FB-CS, or EC-CS) were combined are illustrated in FIGS. 19 and 20. In FIGS. 19 and 20, cutoffs for AI-powered tumor microenvironment factors were determined as median values of the AI-H3 prop≥50% group as indicated in plots.

FIG. 19 illustrates Kaplan-Meier curves showing progression free survival (PFS) according to the quantitative proportion (AI-H3 prop) of tumor cells exhibiting AI-powered HER2 IHC 3+ (H3) staining intensity and AI-powered tumor microenvironment factors.

FIG. 19A is a Kaplan-Meier curve for AI-H3 prop and the lymphocyte density of a cancer stroma region (LC-CS), FIG. 19B is a Kaplan-Meier curve for AI-H3 prop and the macrophage density of a cancer stroma region (MP-CS), FIG. 19C is a Kaplan-Meier curve for AI-H3 prop and the fibroblast density of a cancer stroma region (FB-CS), and FIG. 19D is a Kaplan-Meier curve for AI-H3 prop and endothelial cell density (EC) in a cancer stroma region (EC-CS).

FIG. 20 illustrates Kaplan-Meier curves showing overall survival (OS) according to the quantitative proportion (AI-H3 prop) of tumor cells exhibiting AI-powered HER2 IHC 3+ (H3) staining intensity and AI-powered tumor microenvironment factors.

FIG. 20A is a Kaplan-Meier curve for AI-H3 prop and the lymphocyte density of a cancer stroma region (LC-CS), FIG. 20B is a Kaplan-Meier curve for AI-H3 prop and the macrophage density of a cancer stroma region (MP-CS), FIG. 20C is a Kaplan-Meier curve for AI-H3 prop and the fibroblast density of a cancer stroma region (FB-CS), and FIG. 20D is a Kaplan-Meier curve for AI-H3 prop and endothelial cell density (EC) in a cancer stroma region (EC-CS).

As illustrated in FIG. 19, the median values of LC-CS, MP-S, FB-CS, and EC-CS were 766.3, 26.4, 1790.8, and 88.3, respectively, and when the median values were used as references, patients with values that are greater than or equal to the median values showed a tendency for PFS to worsen. However, the other cases except for FB-CS (HR 2.86, 95% CI 1.02-8.02, p=0.046) were not statistically significant. In addition, as illustrated in FIG. 20, a similar pattern was observed for OS, which was also not statistically significant.

### Example 8. Clinical Outcome Analysis of HER2-Targeted Therapy According to AI-Powered Tumor Microenvironment Factors when Quantitative Proportion of Tumor Cells Exhibiting AI-Powered HER2 IHC 3+ (H3) Staining Intensity is 50% or More (AI-H3 Prop≥50%)

### 8-1. Analysis of Hazard Ratio for Each AI-Powered Tumor Microenvironment Factor

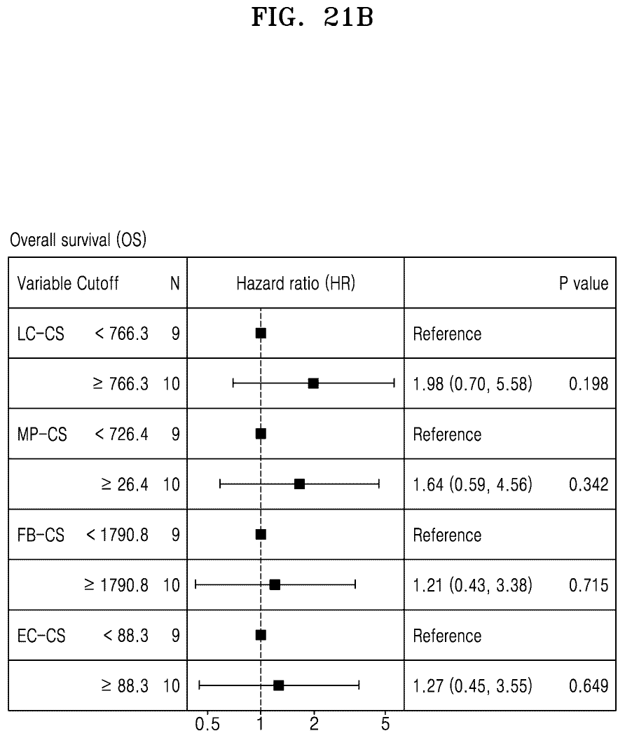

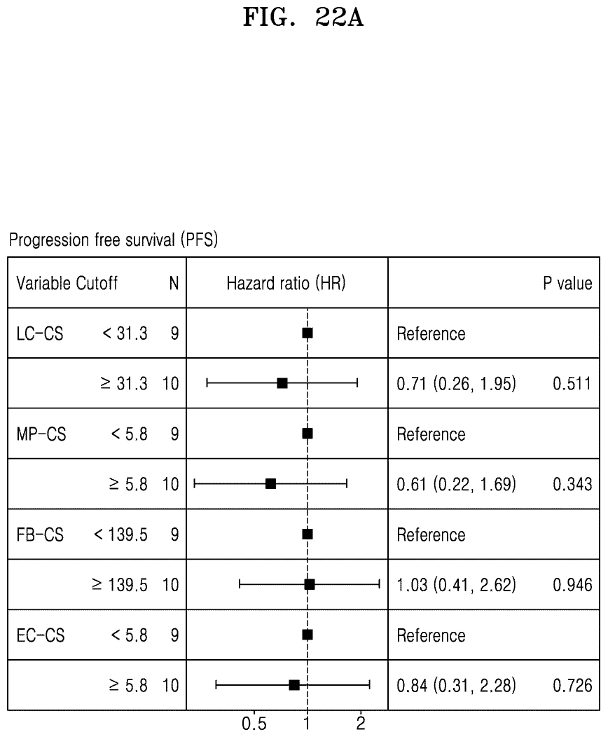

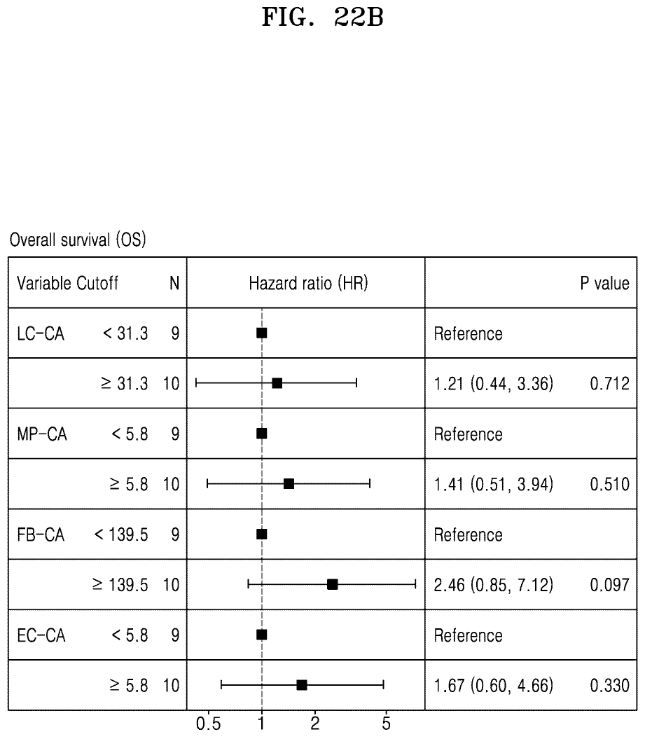

Based on the results of determining median progression-free survival (mPFS) and median overall survival (mOS) and 95% confidence intervals (95 Cls) by using Kaplan-Meier curves as described in Example 7, the hazard ratios (HR) and p-values of the 95% confidence intervals for each AI-analyzed tumor microenvironment (TME) factor were calculated through Cox regression analysis, and the results thereof are illustrated in FIGS. 21 and 22.

FIG. 21 illustrates graphs showing hazard ratios (HR) according to lymphocyte density (LC-CS), fibroblast density (FB-CS), macrophage density (MP-CS), and endothelial cell density (EC-CS), in cancer stroma (CS) regions of patients having a quantitative proportion of tumor cells exhibiting AI-powered HER2 IHC 3+ (H3) staining intensity of 50% or more (AI-H3 prop≥50%).

FIG. 21A is a graph showing the hazard ratio (HR) for PFS in patients having a quantitative proportion of tumor cells exhibiting AI-powered HER2 IHC 3+ (H3) staining intensity of 50% or more (AI-H3 prop≥50%), and FIG. 21B is a graph showing the HR for OS in patients having a quantitative proportion of tumor cells exhibiting AI-powered HER2 IHC 3+ (H3) staining intensity of 50% or more (AI-H3 prop≥50%).

As illustrated in FIG. 21, it was confirmed that distinct trends were observed when stratifying by median values of tumor microenvironment (TME) factors determined through AI-powder TME analysis in a group (N=19) of patients with AI-H3 prop≥50%. Specifically, the median values of LC-CS, MP-S, FB-CS, and EC-CS were 766.3, 26.4, 1790.8, and 88.3, respectively, and when the median values were used as cutoffs, patients with values that were greater than or equal to the median values were confirmed to show poorer PFS. However, the other cases except for FB-CS (HR 2.86, 95% confidence intervals 1.02-8.02, p=0.046) were not statistically significant. In addition, a similar pattern was observed for OS, but this was not statistically significant.

FIG. 22 illustrates graphs showing hazard ratios (HR) according to lymphocyte density (LC-CA), fibroblast density (FB-CA), macrophage density (MP-CA), and endothelial cell density (EC-CA), in cancer areas (CAs) of patients having a quantitative proportion of tumor cells exhibiting AI-powered HER2 IHC 3+ (H3) staining intensity of 50% or more (AI-H3 prop≥50%).

FIG. 22A is a graph showing the hazard ratio (HR) for PFS in patients having a quantitative proportion of tumor cells exhibiting AI-powered HER2 IHC 3+ (H3) staining intensity of 50% or more (AI-H3 prop>50%), and FIG. 22B is a graph showing the HR for OS in patients having a quantitative proportion of tumor cells exhibiting AI-powered HER2 IHC 3+ (H3) staining intensity of 50% or more (AI-H3 prop≥50%).

As illustrated in FIG. 22, in contrast to the hazard ratio (HR) measured in the cancer stroma (CS) area, a group of patients having values that were greater than or equal to the median values of LC-CA, MP-CA, FB-CA, and EC-CA exhibited better PFS, but this was not statistically significant. However, in the case of OS, the group of patients having values that were greater than or equal to the median values exhibited poorer OS, but this was not statistically significant.

This indicates that patients who had a quantitative proportion of tumor cells exhibiting AI-powered HER2 IHC 3+ (H3) staining intensity of 50% or more (AI-H3 prop≥50%) and had a value less than the median value (1790.8) of FB-CS were highly likely to have good drug sensitivity and treatment prognosis for pertuzumab and trastuzumab.

### 8-2. Analysis of Progression-Free Survival (PFS) and Overall Survival (OS) According to Combination of AI-Powered Tumor Microenvironment Factors

For patients with HER2-amplified metastatic colorectal cancer (mCRC) who were administered with pertuzumab and trastuzumab (dual HER2-targeted therapy), the quantitative proportion (AI-H3 prop) of tumor cells exhibiting AI-powered HER2 IHC 3+ (H3) staining intensity and two or three tumor microenvironment (TME) factors derived through TME analysis were combined to perform survival analysis.

Specifically, survival outcomes were compared among the following groups: AI-H3 prop<50%, AI-H3 prop≥50% with all selected TME factors≥median value (TME-high), and AI-H3 prop≥50% with the remaining cases (TME-low). The results of survival analysis for progression-free survival (PFS) are shown in Table 3, and the results of survival analysis for overall survival (OS) are shown in Table 4.

Table 3 shows the results of survival analysis for PFS through combination of two or three tumor microenvironment (TME) factors derived from the AI-powered TME analyzer and the quantitative proportion (AI-H3 prop) of tumor cells exhibiting HER2 IHC 3+ (H3) staining intensity derived as a result of AI-powered HER2 QCS analysis. Median progression-free survival (mPFS) and 95% confidence intervals (Cls) were determined using Kaplan-Meier curves. Hazard ratios (HRs) with 95% Cls and p-values were calculated through Cox regression analysis. The TME factor cutoff for the AI-H3 prop≥50% group was set as the median value.

Specifically, lymphocyte density (LC-CS), macrophage density (MP-CS), fibroblast density (FB-CS) and endothelial cell density (EC-CS) in the cancer stroma (CS) area were 766.3, 26.4, 1790.8, and 88.3, respectively, “N” represents the number of patients of each group, “NR” represents not reached, and “Ref.” represents a reference group.

TABLE 3

| TME | | | mPFS | HR | P |
|---|---|---|---|---|---|
| factors | Group | N | (95% CI) | (95% CI) | value |
| LC-CS, | Al—H3 prop < 50% | 11 | 1.41 (1.25-NR) | 9.88 (3.02-32.32) | <0.001 |
| MP-CS | Al—H3 prop ≥ 50% & TME-high | 8 | 3.10 (1.31-NR) | 1.69 (0.65-4.42) | 0.284 |
| | Al—H3 prop ≥ 50% & TME-low | 11 | 5.59 (4.44-NR) | Ref. | Ref. |
| LC-CS, | Al—H3 prop < 50% | 11 | 1.41 (1.25-NR) | 32.27 (5.98-174.05) | <0.001 |
| FB-CS | Al—H3 prop ≥ 50% & TME-high | 6 | 1.77 (1.28-NR) | 15.81 (3.12-80.11) | <0.001 |
| | Al—H3 prop ≥ 50% & TME-low | 13 | 5.59 (4.44-NR) | Ref. | Ref. |
| LC-CS, | Al—H3 prop < 50% | 11 | 1.41 (1.25-NR) | 12.11 (3.52-41.63) | <0.001 |
| EC-CS | Al—H3 prop ≥ 50% & TME-high | 9 | 3.98 (1.31-NR) | 2.29 (0.89-5.87) | 0.085 |
| | Al—H3 prop ≥ 50% & TME-low | 10 | 5.59 (4.44-NR) | Ref. | Ref. |
| MP-CS, | Al—H3 prop < 50% | 11 | 1.41 (1.25-NR) | 10.81 (3.29-35.47) | <0.001 |
| FB-CS | Al—H3 prop ≥ 50% & TME-high | 7 | 2.23 (1.28-NR) | 2.28 (0.84-6.17) | 0.105 |
| | Al—H3 prop ≥ 50% & TME-low | 12 | 5.57 (4.27-NR) | Ref. | Ref. |
| MP-CS, | Al—H3 prop < 50% | 11 | 1.41 (1.25-NR) | 10.23 (3.12-33.58) | <0.001 |
| EC-CS | Al—H3 prop ≥ 50% & TME-high | 8 | 3.10 (1.31-NR) | 1.82 (0.71-4.70) | 0.215 |
| | Al—H3 prop ≥ 50% & TME-low | 11 | 5.59 (4.44-NR) | Ref. | Ref. |
| FB-CS, | Al—H3 prop < 50% | 11 | 1.41 (1.25-NR) | 13.21 (3.86-45.17) | <0.001 |
| EC-CS | Al—H3 prop ≥ 50% & TME-high | 7 | 2.23 (1.28-NR) | 3.40 (1.24-9.33) | 0.018 |
| | Al—H3 prop ≥ 50% & TME-low | 12 | 5.59 (4.44-NR) | Ref. | Ref. |
| LC-CS, | Al—H3 prop < 50% | 11 | 1.41 (1.25-NR) | 22.99 (5.31-99.62) | <0.001 |
| MP-CS, | Al—H3 prop ≥ 50% & TME-high | 5 | 1.31 (1.28-NR) | 14.34 (3.35-61.44) | <0.001 |
| FB-CS | Al—H3 prop ≥ 50% & TME-low | 14 | 5.59 (4.44-20.17) | Ref. | Ref. |
| LC-CS, | Al—H3 prop < 50% | 11 | 1.41 (1.25-NR) | 11.25 (3.41-37.08) | <0.001 |
| MP-CS, | Al—H3 prop ≥ 50% & TME-high | 7 | 2.23 (1.28-NR) | 2.48 (0.93-6.63) | 0.07 |
| EC-CS | Al—H3 prop ≥ 50% & TME-low | 12 | 5.59 (4.44-NR) | Ref. | Ref. |
| LC-CS, | Al—H3 prop < 50% | 11 | 1.41 (1.25-NR) | 32.27 (5.98-174.05) | <0.001 |
| FB-CS, | Al—H3 prop ≥ 50% & TME-high | 6 | 1.77 (1.28-NR) | 15.81 (3.12-80.11) | <0.001 |
| EC-CS | Al—H3 prop ≥ 50% & TME-low | 13 | 5.59 (4.44-NR) | Ref. | Ref. |
| MP-CS, | Al—H3 prop < 50% | 11 | 1.41 (1.25-NR) | 11.58 (3.54-37.88) | <0.001 |
| FB-CS, | Al—H3 prop ≥ 50% & TME-high | 6 | 1.77 (1.28-NR) | 3.13 (1.12-8.73) | 0.029 |
| EC-CS | Al—H3 prop ≥ 50% & TME-low | 13 | 5.59 (4.27-NR) | Ref. | Ref. |

Table 4 shows the results of survival analysis for overall survival (OS) through combination of two or three TME factors derived from the AI-powered TME analyzer and the quantitative proportion (AI-H3 prop) of tumor cells exhibiting HER2 IHC 3+ (H3) staining intensity derived as a result of AI-powered HER2 QCS analysis.

Median overall survival (mOS) and 95% confidence intervals (Cls) were determined using Kaplan-Meier curves. Hazard ratios (HRs) with 95% Cls and p-values were calculated through Cox regression analysis. The TME factor cutoff for the AI-H3 prop≥50% group was set as the median value. Specifically, lymphocyte density (LC-CS), macrophage density (MP-CS), fibroblast density (FB-CS) and endothelial cell density (EC-CS) in the cancer stroma (CS) area were 766.3, 26.4, 1790.8, and 88.3, respectively, “N” represents the number of patients of each group, “NR” represents not reached, and “Ref.” represents a reference group.

TABLE 4

| TME | | | mOS | HR | P |
|---|---|---|---|---|---|
| factors | Group | N | (95% CI) | (95% CI) | value |
| LC-CS, | Al—H3 prop < 50% | 11 | 4.11 (2.37-NR) | 11.87 (3.58-39.33) | <0.001 |
| MP-CS | Al—H3 prop ≥ 50% & TME-high | 8 | 7.36 (4.50-NR) | 2.82 (1.00-7.92) | 0.049 |
| | Al—H3 prop ≥ 50% & TME-low | 11 | 28.65 (16.49-NR) | Ref. | Ref. |
| LC-CS, | Al—H3 prop < 50% | 11 | 4.11 (2.37-NR) | 12.07 (3.80-38.36) | <0.001 |
| FB-CS | Al—H3 prop ≥ 50% & TME-high | 6 | 5.21 (2.20-NR) | 4.16 (1.45-11.94) | 0.008 |
| | Al—H3 prop ≥ 50% & TME-low | 13 | 23.36 (12.88-NR) | Ref. | Ref. |
| LC-CS, | Al—H3 prop < 50% | 11 | 4.11 (2.37-NR) | 10.25 (3.12-33.67) | <0.001 |
| EC-CS | Al—H3 prop ≥ 50% & TME-high | 9 | 8.80 (4.50-NR) | 1.96 (0.71-5.44) | 0.196 |
| | Al—H3 prop ≥ 50% & TME-low | 10 | 22.54 (12.42-NR) | Ref. | Ref. |
| MP-CS, | Al—H3 prop < 50% | 11 | 4.11 (2.37-NR) | 10.53 (3.33-33.34) | <0.001 |
| FB-CS | Al—H3 prop ≥ 50% & TME-high | 7 | 5.91 (2.20-NR) | 2.57 (0.91-7.29) | 0.076 |
| | Al—H3 prop ≥ 50% & TME-low | 12 | 25.18 (12.88-NR) | Ref. | Ref. |
| MP-CS, | Al—H3 prop < 50% | 11 | 4.11 (2.37-NR) | 9.15 (2.91-28.81) | <0.001 |
| EC-CS | Al—H3 prop ≥ 50% & TME-high | 8 | 7.36 (4.50-NR) | 1.69 (0.60-4.78) | 0.325 |
| | Al—H3 prop ≥ 50% & TME-low | 11 | 23.36 (16.49-NR) | Ref. | Ref. |
| FB-CS, | Al—H3 prop < 50% | 11 | 4.11 (2.37-NR) | 9.68 (3.11-30.15) | <0.001 |
| EC-CS | Al—H3 prop ≥ 50% & TME-high | 7 | 5.91 (2.20-NR) | 2.13 (0.74-6.07) | 0.159 |
| | Al—H3 prop ≥ 50% & TME-low | 12 | 22.54 (12.88-NR) | Ref. | Ref. |
| LC-CS, | Al—H3 prop < 50% | 11 | 4.11 (2.37-NR) | 51.97 (6.35-425.62) | <0.001 |
| MP-CS, | Al—H3 prop ≥ 50% & TME-high | 5 | 4.50 (2.20-NR) | 55.98 (5.89-531.74) | <0.001 |
| FB-CS | Al—H3 prop ≥ 50% & TME-low | 14 | 26.00 (16.49-NR) | Ref. | Ref. |
| LC-CS, | Al—H3 prop < 50% | 11 | 4.11 (2.37-NR) | 11.38 (3.53-36.67) | <0.001 |
| MP-CS, | Al—H3 prop ≥ 50% & TME-high | 7 | 5.91 (2.20-NR) | 3.01 (1.05-8.62) | 0.04 |
| EC-CS | Al—H3 prop ≥ 50% & TME-low | 12 | 26.00 (16.49-NR) | Ref. | Ref. |
| LC-CS, | Al—H3 prop < 50% | 11 | 4.11 (2.37-NR) | 12.07 (3.80-38.36) | <0.001 |
| FB-CS, | Al—H3 prop ≥ 50% & TME-high | 6 | 5.21 (2.20-NR) | 4.16 (1.45-11.94) | 0.008 |
| EC-CS | Al—H3 prop ≥ 50% & TME-low | 13 | 23.36 (12.88-NR) | Ref. | Ref. |
| MP-CS, | Al—H3 prop < 50% | 11 | 4.11 (2.37-NR) | 10.53 (3.38-32.80) | <0.001 |
| FB-CS, | Al—H3 prop ≥ 50% & TME-high | 6 | 5.21 (2.20-NR) | 3.08 (1.03-9.21) | 0.044 |
| EC-CS | Al—H3 prop ≥ 50% & TME-low | 13 | 23.36 (12.88-NR) | Ref. | Ref. |

As shown in Tables 3 and 4, the AI-H3 prop<50% group exhibited significantly poorer PFS and OS than the AI-H3 prop≥50% group with TME-low. In particular, it was confirmed that the largest HR for PFS was shown in the case of a combination of LC-CS and FB-CD or the case of a combination of LC-CS, FB-CS, and EC-CS (HR 32.27, 95% CI 5.98-174.05, p<0.001). It was also confirmed that the highest HR for OS was shown in the case of a combination of LC-CS, MP-CS, and FB-CS (HR 51.97, 95% CI, 6.35-425.62, p<0.001).

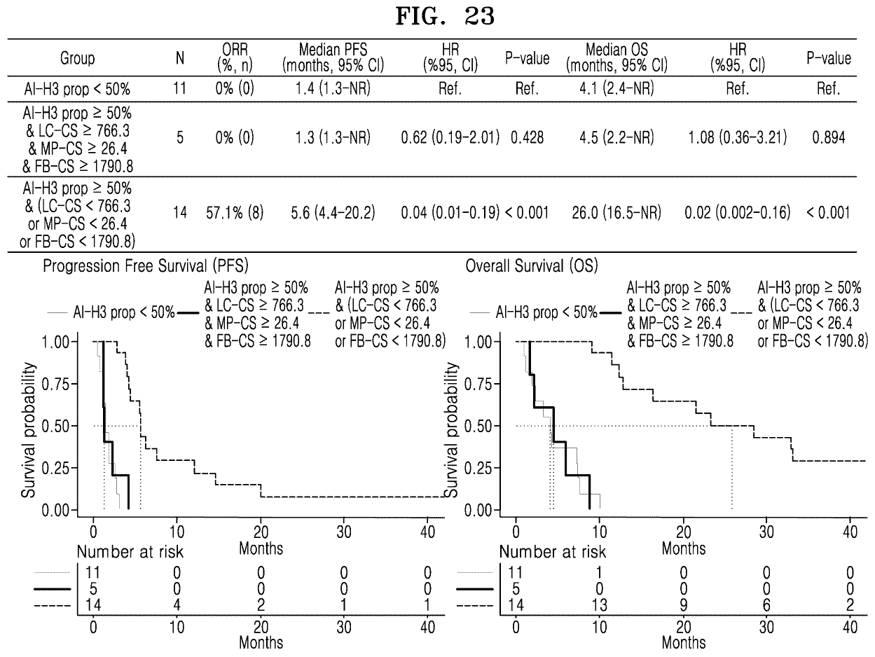

Kaplan-Meier curves showing the progression-free survival (PFS) and overall survival (OS) of the combination of LC-CS, MP-CS, and FB-CS were illustrated in FIG. 23.

FIG. 23 illustrates Kaplan-Meier curves showing the progression-free survival (PFS) and overall survival (OS) of a combination of LC-CS, MP-CS, and FB-CS.

The median progression-free survival (mPFS) was 1.4 months (95% CI, 1.3—not reached) for AI-H3 prop<50%, 1.3 months (95% CI, 1.3—not reached) for AI-H3 prop≥50% with TME-high, and 5.6 months (95% CI, 4.4-20.2) for AI-H3 prop≥50% with TME-low.

As illustrated in FIG. 23, the HR for PFS in the AI-H3 prop≥50% with TME-high group was not significantly different when compared with the AI-H3 prop<50% group (HR 0.62, 95% CI, 0.19-2.01, p=0.428). However, the HR for PFS in the group of AI-H3 prop≥50% with TME-low was significantly higher than that in the group of AI-H3 prop<50% (HR 0.04, 95% CI, 0.01-0.19, p<0.001).

The median overall survival (mOS) was 4.1 months (95% CI, 2.4—not reached) for AI-H3 prop<50%, 4.5 months (95% CI, 2.2—not reached) for AI-H3 prop≥50% with TME-high, and 26.0 months (95% CI, 16.5—not reached) for AI-H3 prop≥50% with TME-low. Similar to the above HR analysis results for PFS, the HR for OS in the AI-H3 prop≥50% with TME-high group did not show a significant difference from that of the AI-H3 prop<50% group (HR 1.08, 95% CI, 0.36-3.21, p=0.894). However, the HR for OS in the group having AI-H3 prop≥50% with TME-low was significantly low, i.e., 0.02 (95% CI, 0.002-0.16, p<0.001). In particular, all cases achieving CR or PR were included in the group having AI-H3 prop≥50% with TME-low, resulting in an ORR of 57.1% (8/14).

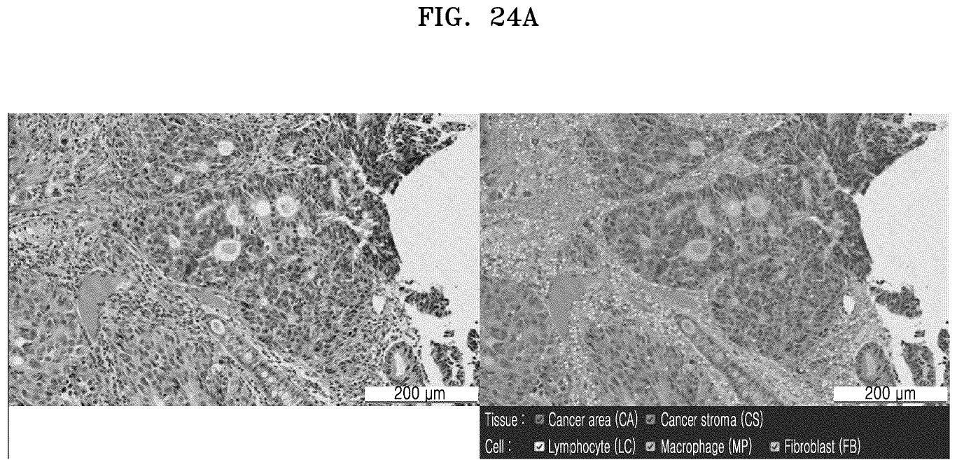

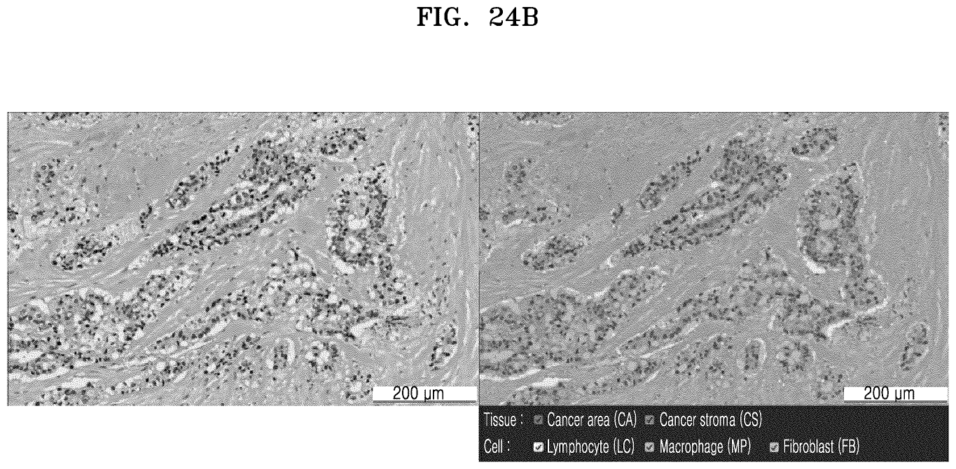

FIG. 24 illustrates representative clinical cases of a group with TME-high or TME-low among groups having a proportion of tumor cells exhibiting HER2 IHC 3+ (H3) staining intensity of 50% or more (AI-H3 prop≥50%) in the TRIUMPH cohort.

FIG. 24A illustrates a representative clinical case exhibiting a PD treatment response in an AI-H3 prop≥50% with TME-high group, and FIG. 24B illustrates a representative clinical case exhibiting a CR treatment response in an AI-H3 prop≥50% with TME-low group.

The left panels represent whole slide images (WSIs), and the right panels represent WSIs processed by an AI analyzer. Each type of cell in the tumor microenvironment (TMW) is indicated by colored dots (yellow: lymphocytes [LC], green: macrophages [MP], orange: fibroblasts [FB]). In addition, the green shaded area represents the cancer area (CA) and the blue shaded area represents the cancer stroma (CS) area.

As illustrated in FIG. 24, the AI-H3 prop≥50% with TME-high group was confirmed to have poor drug sensitivity and treatment prognosis (PD) for pertuzumab and trastuzumab in actual clinical outcomes, and the AI-H3 prop≥50% with TME-low group was confirmed to have good treatment prognosis (CR) in actual clinical outcomes.

### 8-3. Analysis of 12- and 24-Month Overall Survival (OS) According to Combination of AI-Powered Tumor Microenvironment Factors

For patients with HER2-amplified metastatic colorectal cancer (mCRC) who were administered with pertuzumab and trastuzumab (dual HER2-targeted therapy), a nomogram for estimating 12-month and 24-month overall survival (OS) rates of patients who had a quantitative proportion (AI-H3 prop) of tumor cells exhibiting AI-powered HER2 IHC 3+ (H3) staining intensity of 50% or more (AI-H3 prop≥50%) was created.

Specifically, the TME factors of each patient with AI-H3 prop≥50% were placed on the corresponding axis, the scores given to each factor were summed, and then the total score was applied to the survival probability scale at the bottom. The TME factors incorporated in the monogram are lymphocyte density (LC-CS), macrophage density (MP-CS), fibroblast density (FB-CS) and endothelial cell density (EC-CS) in the carbon stroma (CS).

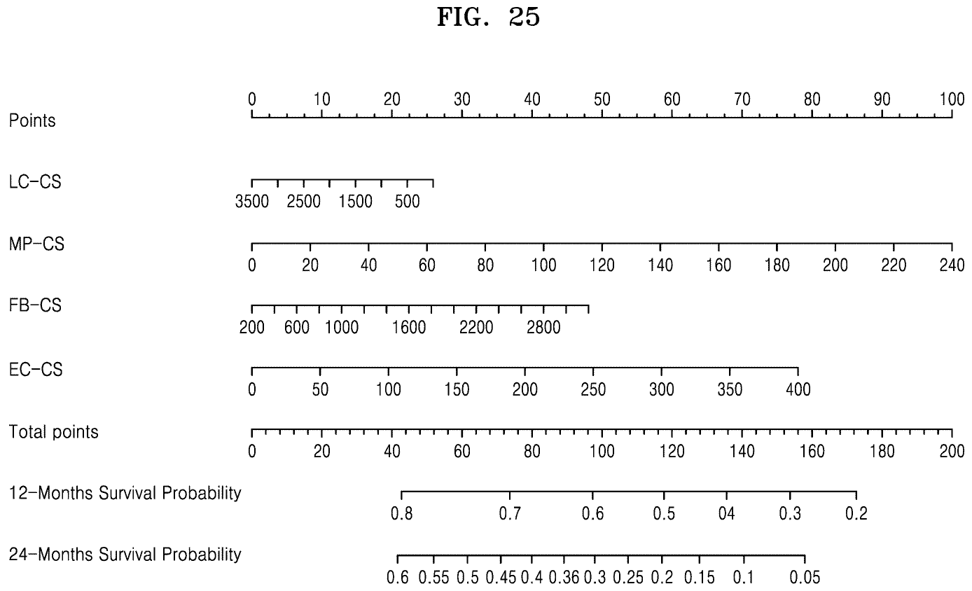

FIG. 25 is a nomogram for estimating 12-month and 24-month overall survival (OS) rates of patients having a proportion of tumor cells exhibiting HER2 IHC 3+ (H3) staining intensity of 50% or more (AI-H3 prop≥50%).

As illustrated in FIG. 25, the highest probabilities of survival after 12 months and 24 months were found to be 780, 50, 1500, and 100, respectively, for LC-CS, MP-S, FB-CS, and EC-CS.

This is consistent with the results of Example 8-2, which showed low risk (HR) and high progression-free survival (PFS) and overall survival (OS) in the AI-H3 prop≥50% with TME-low group.

### Example 9. Correlation Analysis Using AI-Powered HER2 QCS Analyzer and AI-Powered TME Analyzer

To analyze the relationship between factors derived through the AI-powered HER2 QCS analyzer and the AI-powered TME analyzer, a total of 144 pairs of HER2 IHC WSIs and hematoxylin and eosin (H&E) WSIs were analyzed in the Prescreening cohort.

Specifically, the factors analyzed in HER2 IHC WSIs using the AI-powered HER2 QCS analyzer may be quantitative proportions (AI-H0 prop, AI-H1 prop, AI-H2 prop, and AI-H3 prop) of tumor cells (TCs) having staining intensity classified according to the expression level of HER2. The factors derived through the AI-powered TME analyzer may be the density of each type of cell in the cancer stroma (CS) area and the cancer area (CA) (LC-CA, MP-CA, FB-CA, and EC-CA/LC-CS, MP-S, FB-CS, and EC-CS).

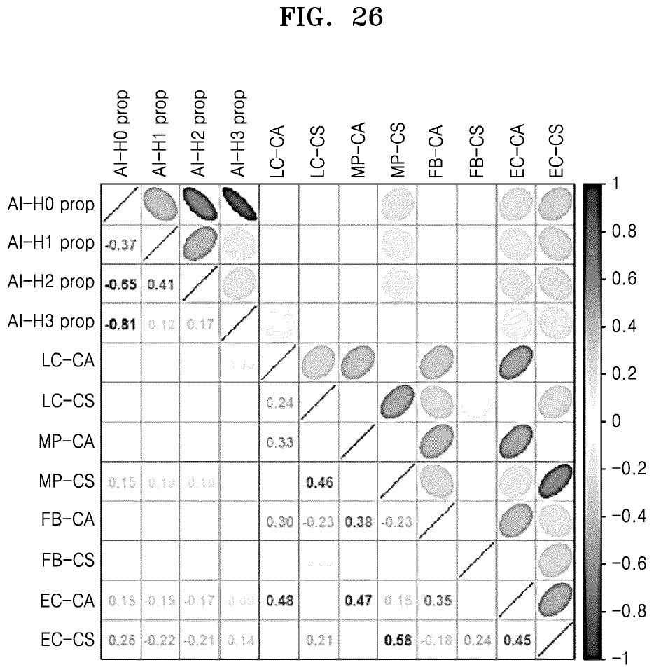

The comprehensive results of the correlation between factors analyzed in HER2 IHC WSIs and H&E WSIs through the AI-powered HER2 QCS analyzer and the AI-powered TME analyzer are shown in FIG. 26.

FIG. 26 is a graph showing the correlation between factors analyzed in HER2 IHC WSIs and H&E WSIs through an AI-powered HER2 QCS analyzer and an AI-powered TME analyzer.

As illustrated in FIG. 26, among the factors analyzed in HER2 IHC WSIs, AI-H3 prop showed a positive correlation with AI-H2 prop (Spearman's p=0.17, p<0.001), and showed a negative correlation with each of AI-H1 prop (p=−0.12, p<0.001) and AI-H0 prop (p=−0.81, p<0.001).

Meanwhile, it was confirmed that there was a positive correlation between the densities of each type of cell in the cancer stroma (CS) area) and each type of cell in the cancer area (CA) among the TME factors analyzed in H&E WSIs. In particular, LC-CS showed a positive correlation with LC-CA (p=0.24, p<0.001), and EC-CS showed a positive correlation with EC-CA (p=0.45, p<0.001).

It was confirmed that AI-H3 prop showed a weak negative correlation with LC-CA, EC-CA, and EC-CS between the factors analyzed in HER2 IHC WSIs and the factors analyzed in H&E WSIs (p=−0.08, −0.09, and −0.14, and p=0.026, 0.001, and 0.017, respectively).

### Example 10. Quantification and Statistical Analysis

F1 score and IoU metrics were used to evaluate the performance of the cell and tissue models. For the evaluation of the agreement between the interpretation by pathologists and the interpretation by AI analyzers, agreement or Cohen's kappa value was used. For categorical variables, comparisons between groups were tested using the chi-squared test or Fisher's exact test. For continuations variables, comparisons between groups were tested using the Student's t-test or nonparametric Mann-Whitney U test.

For correlation analysis, the Pearson correlation coefficient or Spearman's rho (p) was used. For survival analysis, Kaplan-Meier curves were plotted, and the HR was calculated using the Cox proportional hazard model. Two-sided p-values were calculated, and a threshold of p<0.05 indicates statistical significance. All statistical analyses were performed using Python (version 3.7) and R software (version 4.2.3) (R Foundation for Statistical Computing, Vienna, Austria).

1. A method for predicting a treatment response to a HER2 targeted therapeutic agent, the method comprising:
identifying HER2 staining intensities of tumor cells (TCs) in a first pathology slide image of a patient by using a machine learning model;
determining a proportion of tumor cells exhibiting HER2 IHC 3+ (H3) staining intensity on the basis of the identified results; and
predicting a treatment response of the patient to the HER2-targeted therapeutic agent on the basis of the proportion of tumor cells exhibiting HER2 IHC 3+ (H3) staining intensity.

2. The method of claim 1, wherein the HER2 staining intensities of the tumor cells comprises HER2 IHC 0 (H0), HER2 IHC 1+ (H1), HER2 IHC 2+ (H2), or HER2 IHC 3+ (H3).

3. The method of claim 1, wherein the HER2-targeted therapeutic agent comprises at least one drug selected from the group consisting of pertuzumab, trastuzumab, trastuzumab emtansine, lapatinib, neratinib, afatinib, tucatinib, and pyrotinib.

4. The method of claim 1, wherein the prediction of the treatment response of the patient to the HER2-targeted therapeutic agent comprises determining that, when the proportion of tumor cells exhibiting HER2 IHC 3+ (H3) staining intensity is 10% or more, the patient responds to the HER2-targeted therapeutic agent.

5. The method of claim 4, wherein the method comprises determining that, when the proportion of tumor cells exhibiting HER2 IHC 3+ (H3) staining intensity is 50% or more, the patient responds to the HER2-targeted therapeutic agent.

6. The method of claim 1, wherein the first pathology slide image is related to a tissue slide collected from solid cancer tissue.

7. The method of claim 6, further comprising determining whether cancer tissue from which the tissue slide is collected is in a state of HER2 overexpression or gene amplification.

8. The method of claim 6, wherein the solid caner comprises at least one selected from the group consisting of lung cancer, skin cancer, stomach cancer, gastrointestinal cancer, intestinal cancer, colorectal cancer, colon cancer, pancreatic cancer, liver cancer, thyroid cancer, uterine cancer, cervical cancer, ovarian cancer, testicular cancer, prostate cancer, breast cancer, and oral cancer.

9. The method of claim 1, further comprising deriving tumor microenvironment (TME) factors from a second pathology slide image of the patient,
wherein the prediction of the treatment response of the patient to the HER2-targeted therapeutic agent comprises predicting a treatment response to a HER2-targeted therapeutic agent by using the proportion of tumor cells exhibiting HER2 IHC 3+ (H3) staining intensity and the TME factors.

10. The method of claim 9, wherein the TME factors are measured in a cancer area (CA) or a cancer stroma (CS) area.

11. The method of claim 9, wherein the TME factor comprise at least one selected from the group consisting of lymphocyte (LC) density, macrophage (MP) density, fibroblast (FB) density, and endothelial cell (EC) density.

12. The method of claim 9, wherein the predicting comprises determining that, when the proportion of tumor cells exhibiting HER2 IHC 3+ (H3) staining intensity is 50% or more and at least one of the TME factors measured in the cancer stroma (CS) area has a value smaller than the following value, the patient responds to the HER2-targeted therapeutic agent.
Lymphocyte (LC) density: 766.3
Macrophage (MP) density: 26.4
Fibroblast (FB) density: 1790.8
Endothelial cell (EC) density: 88.3.

13. A computer program stored on a computer-readable recording medium for executing the method according to claim 1 on a computer.

14. A computing system comprising:
at least one memory; and
at least one processor connected to the at least one memory and configured to execute at least one computer-readable program included in the at least one memory,
wherein the at least one processor is configured to, by executing the at least one program,
identify HER2 staining intensities of tumor cells (TCs) in a first pathology slide image of a patient by using a machine learning model, determine a proportion of tumor cells exhibiting the HER2 IHC 3+ (H3) staining intensity on the basis of the identified results, and predict a treatment response of the patient to a HER2-targeted therapeutic agent on the basis of the proportion of tumor cells exhibiting the HER2 IHC 3+ (H3) staining intensity.

15. The computing system of claim 14, wherein the processor is further configured to derive tumor microenvironment (TME) factors from a second pathology slide image of the patient, and predict a treatment response to a HER2-targeted therapeutic agent by using the proportion of tumor cells exhibiting the HER2 IHC 3+ (H3) staining intensity and the TME factors.

16. The computing system of claim 14, wherein the processor is configured to determine that, when the proportion of tumor cells exhibiting the HER2 IHC 3+ (H3) staining intensity is 50% or more, the patient respond to the HER2-targeted therapeutic agent.

17. The computing system of claim 14, wherein the processor is configured to determine that, when the proportion of tumor cells exhibiting the HER2 IHC 3+ (H3) staining intensity is 50% or more and at least one of the TME factors measured in a cancer stroma (CS) area has a value smaller than the following value, the patient responds to the HER2-targeted therapeutic agent.
Lymphocyte (LC) density: 766.3
Macrophage (MP) density: 26.4
Fibroblast (FB) density: 1790.8
Endothelial cell (EC) density: 88.3.

18. A method of treating cancer, the method comprising:
identifying HER2 staining intensities of tumor cells (TCs) in a first pathology slide image of a patient by using a machine learning model;
determining a proportion of tumor cells exhibiting HER2 IHC 3+ (H3) staining intensity on the basis of the identified results;
predicting a treatment response of the patient to a HER2-targeted therapeutic agent on the basis of the proportion of tumor cells exhibiting HER2 IHC 3+ (H3) staining intensity; and
administering the HER2-targeted therapeutic agent to the patient, when it is determined that the patient responds to the HER2-targeted therapeutic agent, on the basis of results of the prediction of the treatment response.

19. The method of claim 18, wherein the prediction of the treatment response of the patient to the HER2-targeted therapeutic agent comprises determining that, when the proportion of tumor cells exhibiting HER2 IHC 3+ (H3) staining intensity is 10% or more, the patient responds to the HER2-targeted therapeutic agent.

20. The method of claim 18, wherein the prediction of the treatment response of the patient to a HER2 targeted therapeutic agent comprises determining that, when the proportion of tumor cells exhibiting HER2 IHC 3+ (H3) staining intensity is 50% or more and at least one of the tumor microenvironment (TME) factors measured in a cancer stroma (CS) area has a value smaller than the following value, the patient responds to the HER2-targeted therapeutic agent.
Lymphocyte (LC) density: 766.3
Macrophage (MP) density: 26.4
Fibroblast (FB) density: 1790.8
Endothelial cell (EC) density: 88.3.
